SEQUENCE LISTING (4 sequences)

SEQ ID NO: 1 - protein, 29 aa, Homo sapiens
HSQGTFTSDYSKYLDSRRAQDFVQWLMNT

SEQ ID NO: 2 - protein, 30 aa, Homo sapiens
HAEGTFTSDVSSYLEGQAAKEFIAWLVKGR

SEQ ID NO: 3 - protein, 31 aa, Homo sapiens
HAEGTFTSDVSSYLEGQAAKEFIAWLVKGRG

SEQ ID NO: 4 - protein, 30 aa, Artificial Sequence
  chimeric GLP-1/glucagon peptide
  MISC_FEATURE (10)..(10): Lysine with a palmitoyl group conjugated to the epsilon nitrogen,through a gamma glutamic acid linker
HSQGTFTSDKSEYLDSERARDFVAWLEAGG

(19) United States
(12) Patent Application Publication
(10) Pub. No.: US 2021/0213104 A1
(43) Pub. Date: Jul. 15, 2021

(54) GLUCAGON AND GLP-1 CO-AGONISTS FOR THE TREATMENT OF CHRONIC KIDNEY DISEASE AND DIABETIC KIDNEY DISEASE IN TYPE 2 DIABETES

(71) Applicant: MEDIMMUNE LIMITED, CAMBRIDGE (GB)

(72) Inventors: VICTORIA PARKER, CAMBRIDGE (GB); DARREN ROBERTSON, CAMBRIDGE (GB); LUTZ JERMUTUS, CAMBRIDGE (GB)

(21) Appl. No.: 17/144,337

(22) Filed: Jan. 8, 2021

Related U.S. Application Data
(60) Provisional application No. 63/089,386, filed on Oct. 8, 2020.
(60) Provisional application No. 63/037,832, filed on Jun. 11, 2020.
(60) Provisional application No. 62/959,698, filed on Jan. 10, 2020.

(51) Int. Cl. A61K 38/26; A61P 13/12; A23L 33/00

(52) U.S. Cl. CPC A61K 38/26; A23L 33/30; A61P 13/12

(57) ABSTRACT

Provided herein are methods of improving glycemic control, reducing weight, decreasing urine albumin:creatinine ratio (UACR) and/or treating diabetic kidney disease (DKD) in a human patient with DKD and type 2 diabetes mellitus (T2DM) comprising administering a GLP-1/glucagon agonist peptide such as Cotadutide. Provided herein are also methods of improving glycemic control, reducing weight, decreasing urine albumin:creatinine ratio (UACR) and/or treating chronic kidney disease (CKD) in a human patient comprising administering a GLP-1/glucagon agonist peptide such as Cotadutide.

### REFERENCE TO SEQUENCE LISTING SUBMITTED ELECTRONICALLY

The content of the electronically submitted sequence listing in ASCII text file (Name: SequenceListing.txt; Size: 1,809 bytes; and Date of Creation: Oct. 8, 2020) filed with the application is incorporated herein by reference in its entirety.

### BACKGROUND

The incidence of obesity and diabetes have been rising in epidemic proportions. Diabetes is characterized by high levels of blood glucose resulting from defects in insulin production, insulin action, or both. Type 2 diabetes mellitus (T2DM) accounts for some 90 to 95 percent of all diagnosed cases of diabetes, and the risk of type 2 diabetes rises with increasing body weight. The prevalence of T2DM is three to seven times higher in those who are affected by obesity than in normal weight adults, and is 20 times more likely in those with a body mass index (BMI) greater than 35 kg/m². In many cases of T2DM, significant weight loss (typically 5% of body weight or more) can promote improvements in glycemic control, cardiovascular risk, and mortality rates. Many existing therapies for T2DM focus on lowering blood glucose. However, there is a major unmet need for treatments that improve glycemic control and achieve disease-modifying weight loss. Renal impairment attributed to chronic hyperglycemia and hypertension is common in patients with longstanding T2DM, and it is often exacerbated by obesity.

The prevalence of chronic kidney disease (CKD) is increasing in parallel with the escalating prevalence of T2DM and obesity as well as aging populations. Kidney diseases are the ninth most common cause of death in high income countries globally. Diabetic kidney disease (DKD) is the most common cause of CKD, accounting for 30-50% of cases and impacting more than 280 million patients globally.

Because dedicated treatments for DKD are limited, a large unmet need exists for this patient group. These patients stand to benefit from medications that can deliver both improvements in glycemic control and weight loss. Treatments for CKD in patients without diabetes, as well as patients with diabetes, are also needed.

### BRIEF SUMMARY

Provided herein are methods of improving glycemic control, reducing weight, decreasing urine albumin:creatinine ratio (UACR) and/or treating chronic kidney disease (CKD) in a human patient with CKD. The methods comprise administering to the patient an effective amount of a GLP-1/glucagon agonist peptide (e.g., Cotadutide (SEQ ID NO:4)).

In some aspects, a method of treating CKD in a human patient comprises administering to the patient a sufficient amount of Cotadutide (SEQ ID NO:4) to treat DKD.

In some aspects, a method of decreasing urine albumin:creatinine ratio (UACR) in a human patient with CKD comprises administering to the patient a sufficient amount of Cotadutide (SEQ ID NO:4) to decrease UACR.

In some aspects, a method of reducing body weight in a human patient with CKD comprises administering to the patient a sufficient amount of Cotadutide (SEQ ID NO:4) to reduce body weight.

In some aspects, a method of improving glycemic control in a human patient with CKD comprises administering to the patient a sufficient amount of Cotadutide (SEQ ID NO:4) to improve glycemic control.

In some aspects, the human patient with CKD has diabetes. In some aspects, the diabetes is type 2 diabetes. In some aspects, the human patient with CKD does not have diabetes.

Provided herein are methods of improving glycemic control, reducing weight, decreasing urine albumin:creatinine ratio (UACR) and/or treating diabetic kidney disease (DKD) in a human patient with DKD or renal insufficiency and type 2 diabetes mellitus (T2DM). The methods comprise administering to the patient an effective amount of a GLP-1/glucagon agonist peptide (e.g., Cotadutide (SEQ ID NO:4)).

In some aspects, a method of treating diabetic kidney disease (DKD) in a human patient with type 2 diabetes mellitus (T2DM) comprises administering to the patient a sufficient amount of Cotadutide (SEQ ID NO:4) to treat DKD.

In some aspects, a method of decreasing urine albumin:creatinine ratio (UACR) in a human patient with T2DM and DKD comprises administering to the patient a sufficient amount of Cotadutide (SEQ ID NO:4) to decrease UACR.

In some aspects, a method of reducing body weight in a human patient with T2DM and DKD comprises administering to the patient a sufficient amount of Cotadutide (SEQ ID NO:4) to reduce body weight.

In some aspects, a method of improving glycemic control in a human patient with T2DM and DKD comprises administering to the patient a sufficient amount of Cotadutide (SEQ ID NO:4) to improve glycemic control.

In some aspects, which can be combined with any other aspects provided herein, the cotadutide is administered at an initial dose of at least 20 μg daily, optionally a dose of about 50 μg daily, and then administered at a second higher dose.

In some aspects, which can be combined with any other aspects provided herein, the cotadutide is administered at a third dose after the administration of the second dose, wherein the third dose is higher than the second dose, optionally wherein the third dose does not exceed 600 μg daily or wherein the third dose does not exceed 300 μg daily.

In some aspects, which can be combined with any other aspects provided herein, the cotadutide is administered at a third dose after the administration of the second dose, optionally a fourth dose after the administration of the third dose, and optionally a fifth dose after the fourth dose, wherein the third dose is higher than the second dose, the fourth dose, when present, is higher than the third dose, the fifth dose, when present, is higher than the fifth dose, and the sixth dose, when present, is higher than the fourth dose.

In some aspects, which can be combined with any other aspects provided herein, the initial dose is administered daily for about 4 days to about 14 days.

In some aspects, which can be combined with any other aspects provided herein, the dose of cotadutide does not exceed 600 μg daily or does not exceed 300 μg daily.

In some aspects, which can be combined with any other aspects provided herein, the cotadutide is administered at an initial dose of 50 μg daily for 14 days and then at a second dose of 100 μg daily. In some aspects, which can be combined with any other aspects provided herein, the cotadutide is administered at the second dose of 100 μg daily for 14-28 days (e.g., 14 days, 21 days, or 28 days) and then at a third dose of 200 μg daily. In some aspects, which can be combined with any other aspects provided herein, the cotadutide is administered at the third dose of 200 μg daily for 14 days and then at a fourth dose of 400 μg daily. In some aspects, which can be combined with any other aspects provided herein, the cotadutide is administered at the fourth dose of 400 μg daily for 14 days and then at a fifth dose of 600 μg daily.

In some aspects, which can be combined with any other aspects provided herein, the cotadutide is administered at an initial dose of 50 μg daily for 4 days and then at a second dose of 100 μg daily. In some aspects, which can be combined with any other aspects provided herein, the cotadutide is administered at the second dose of 100 μg daily for 7 days and then at a third dose of 200 μg daily. In some aspects, which can be combined with any other aspects provided herein, the cotadutide is administered at the third dose of 200 μg daily and then at a fourth dose of 300 μg daily.

In some aspects, which can be combined with any other aspects provided herein, the cotadutide is administered at an initial dose of 100 μg daily for 7 days, at second dose of 200 μg daily for the next 7 days, and subsequently at a dose of 300 μg daily.

In some aspects, which can be combined with any other aspects provided herein, the cotadutide is administered by injection, optionally wherein the administration is subcutaneous.

In some aspects, which can be combined with any other aspects provided herein, the administration reduces the mixed-meal tolerance test (MMTT) plasma glucose area under the curve (AUC)₀₋₄ hours in the patient. The reduction can occur with 3 weeks, within 12 weeks, within 14 weeks, or within 26 weeks from the initial administration of the Cotadutide. In some aspects, the administration reduces the MMTT plasma glucose AUC by at least 15%, e.g., within 32 days. In some aspects, the administration reduces the MMTT plasma glucose AUC by at least 20%, e.g., within 32 days. In some aspects, the administration reduces the MMTT plasma glucose AUC by at least 25%, e.g., within 32 days. In some aspects, the administration reduces the MMTT plasma glucose AUC by 15% to 30%, 20% to 30%, or 25% to 30%, e.g., within 32 days. In some aspects, the administration reduces the MMTT plasma glucose AUC by 15% to 40%, 20% to 40%, or 25% to 40%, e.g., within 32 days. In some aspects, the administration reduces the MMTT plasma glucose AUC by 15% to 50%, 20% to 50%, or 25% to 50%, e.g., within 32 days.

In some aspects, which can be combined with any other aspects provided herein, the administration reduces Hemoglobin A1c (HbA1c) in the patient. The reduction can occur with 3 weeks, within 12 weeks, within 14 weeks, or within 26 weeks from the initial administration of the Cotadutide.

In some aspects, which can be combined with any other aspects provided herein, the administration reduces fasting plasma glucose (FPG) in the patient. The reduction can occur with 3 weeks, within 12 weeks, within 14 weeks, or within 26 weeks from the initial administration of the Cotadutide.

In some aspects, which can be combined with any other aspects provided herein, the administration reduces continuous glucose monitoring (CGM) glucose AUC₀₋₂₄ in the patient. The reduction can occur with 3 weeks, within 12 weeks, within 14 weeks, or within 26 weeks from the initial administration of the Cotadutide.

In some aspects, which can be combined with any other aspects provided herein, the administration reduces hyperglycemic glucose levels in the patient. The reduction can occur with 3 weeks, within 12 weeks, within 14 weeks, or within 26 weeks from the initial administration of the Cotadutide.

In some aspects, which can be combined with any other aspects provided herein, the administration reduces insulin use by the patient. The reduction can occur with 3 weeks, within 12 weeks, within 14 weeks, or within 26 weeks from the initial administration of the Cotadutide.

In some aspects, which can be combined with any other aspects provided herein, the administration increases the amount of time the patient has euglycemic glucose levels. The amount of time can be measured over a 7-day period.

In some aspects, which can be combined with any other aspects provided herein, the administration improves insulin resistance in the patient, optionally wherein the insulin resistance is measured using the Homeostatic Model Assessment of Insulin Resistance (HOMA-IR) and/or the MATSUDA index. In some aspects, which can be combined with any other aspects provided herein, the administration improves beta cell function in the patient.

In some aspects, which can be combined with any other aspects provided herein, the administration treats DKD in the patient. In some aspects, which can be combined with any other aspects provided herein, the administration treats CKD in the patient.

In some aspects, which can be combined with any other aspects provided herein, the administration decreases the urine albumin:creatinine ratio (UACR) of the patient. In some aspects, which can be combined with any other aspects provided herein, the administration decreases the UACR more effectively than semaglutide decreases UACR. In some aspects, the administration decreases the UACR of the patient by at least 40%, e.g., within 32 days. In some aspects, the administration decreases the UACR of the patient by at least 45%, e.g., within 32 days. In some aspects, the administration decreases the UACR of the patient by at least 50%, e.g., within 32 days. In some aspects, the administration decreases the UACR of the patient by 40% to 60%, 45% to 60%, or 50% to 60% e.g., within 32 days. In some aspects, the administration decreases the UACR of the patient by 40% to 75%, 45% to 75%, or 50% to 75% e.g., within 32 days

In some aspects, which can be combined with any other aspects provided herein, the administration reduces body weight of the patient. The body weight can be reduced by at least 3%. The body weight can be reduced by at least 5% or by at least 10%. The body weight can be reduced by 3% to 15%, by 5% to 15%, or by 10% to 15%. The body weight can be reduced by 3% to 20%, by 5% to 20%, or by 10% to 20%. The body weight can be reduced by 3% to 25%, by 5% to 25%, or by 10% to 25%. The body weight can be reduced by 3% to 30%, by 5% to 30%, or by 10% to 30%.

In some aspects, which can be combined with any other aspects provided herein, the administration improves glycemic control in the patient.

In some aspects, which can be combined with any other aspects provided herein, the administration is for at least two weeks, for at least 12 weeks, for at least 14 weeks, or for at least 26 weeks.

In some aspects, which can be combined with any other aspects provided herein, the administration is an adjunct to diet and exercise.

In some aspects, which can be combined with any other aspects provided herein, the patient has an estimated glomerular filtration rate (eGFR) of <90 mL/min/1.73 m² prior to the administration. In some aspects, which can be combined with any other aspects provided herein, the patient has an eGFR <60 mL/min/1.73 m² prior to the administration. In some aspects, which can be combined with any other aspects provided herein, the patient has an eGFR ≥20 mL/min/m² prior to the administration. In some aspects, which can be combined with any other aspects provided herein, the patient has an eGFR ≥30 mL/min/m² prior to the administration.

In some aspects, which can be combined with any other aspects provided herein, the patient has micro- or macro-albuminuria.

In some aspects, which can be combined with any other aspects provided herein, the patient has an HBA1c<8.0% prior to the administration

In some aspects, which can be combined with any other aspects provided herein, the patient has a body mass index (BMI) of ≥23 kg/m² or ≥25 kg/m² prior to the administration.

In some aspects, which can be combined with any other aspects provided herein, the patient has a BMI ≤40 kg/m² prior to the administration.

In some aspects, which can be combined with any other aspects provided herein, the patient has UACR >3 mg/mmol prior to the administration.

### BRIEF DESCRIPTION OF THE DRAWINGS/FIGURES

FIG. 1 shows the chemical structure, chemical formula (C₁₆₇H₂₅₂N₄₂O₅₅), and molecular weight (3728.09), for Cotadutide (MEDI0382; SEQ ID NO:4).

FIG. 2 provides a flow diagram of a Phase 2a study of Cotadutide (“MEDI0382”) in patients with T2DM and renal impairment. (See Example 2.)

FIG. 3 shows the plasma glucose concentrations (mg/dL) during mixed meal tolerance tests (MMTTs) in the intent-to-treat (ITT) population. (See Example 2.)

FIG. 4 shows the percent change in weight (kg) from Day 1 to Day 60 in the ITT population. (See Example 2.)

FIG. 5 shows the percentage of time spent within various continuous glucose monitoring (CGM) glucose ranges during treatment across 32 days of dosing in the ITT population. (See Example 2.)

FIG. 6 shows the diminishing hypoglycemia in the Cotadutide arm, likely to due to insulin dose adjustment. (See Example 2.)

FIG. 7 shows the mean change of urine albumin:creatinine ratio (UACR) from baseline in subjects with micro- or macro-albuminuria at baseline. (See Example 2.)

FIG. 8 shows the change in eGFR from baseline to day 60 in the ITT population. (See Example 2.)

FIG. 9 shows the Cotadutide geometric mean and individual plasma concentrations at predose (C_{trough}.) (See Example 2.)

FIG. 10 shows the change in total daily insulin dose from Day −2 (baseline) to Day 40 in participants with insulin dose of at least 20 U/day. (See Example 2.)

FIG. 11 provides a flow diagram of a Phase 2b study of Cotadutide in patients with T2DM and diabetic kidney disease (DKD). In addition to the 12-week primary analysis, a 14-week analysis can be completed. (See Example 3.)

### DETAILED DESCRIPTION

### I. Definitions

Throughout this disclosure, the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. A peptide “comprising” a particular amino acid sequence refers to a peptide containing the amino acid sequence, wherein the peptide may or may not contain additional amino acids or other modifications to the amino acid sequence. A peptide “consisting of” a particular amino acid sequence refers to a peptide containing only the amino acid sequence and no additional amino acids or other modifications to the amino acid sequence. A peptide “comprising” an amino acid sequence “consisting of” a particular amino acid sequence refers to a peptide containing the amino acid sequence and no additional amino acids; however, the peptide may comprise other modifications to the amino acid sequence (e.g., an acyl moiety or a palmitoyl moiety).

It is understood that wherever aspects are described herein with the language “about” a number, otherwise analogous aspects referring to the specified number (without “about”) are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, proteolytic cleavage, or modification by non-naturally occurring amino acids.

More specifically, the term “peptide” as used herein encompasses full length peptides and fragments, variants or derivatives thereof, e.g., a GLP-1/glucagon agonist peptide (e.g., 29, 30, or 31 amino acids in length). A “peptide” as disclosed herein, e.g., a GLP-1/glucagon agonist peptide, can be part of a fusion polypeptide comprising additional components such as, e.g., an Fc domain or an albumin domain, to increase half-life. A peptide as described herein can also be derivatized in a number of different ways. A peptide described herein can comprise modifications including e.g., conjugation of a palmitoyl group.

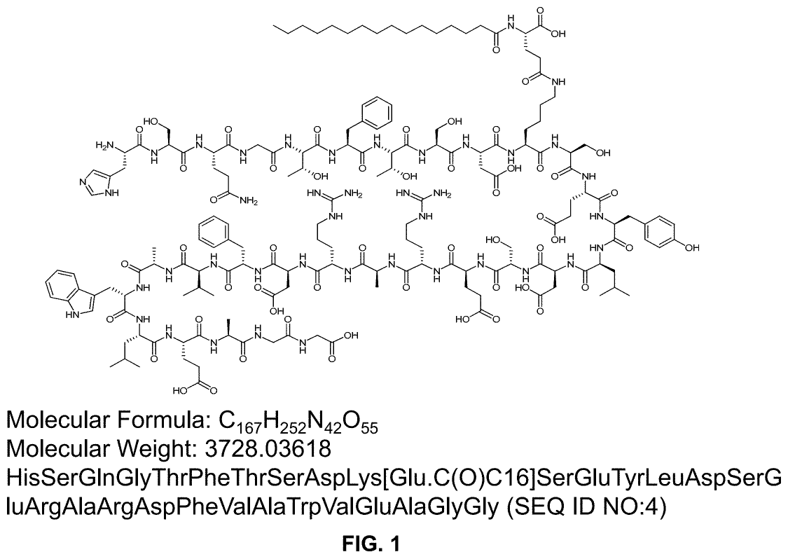

The terms “Cotadutide” and “MEDI0382” are used herein to refer to a peptide with the structure shown in FIG. 1.

The term “isolated” refers to the state in which peptides or nucleic acids, will generally be in accordance with the present disclosure. Isolated peptides and isolated nucleic acids will be free or substantially free of material with which they are naturally associated such as other peptides or nucleic acids with which they are found in their natural environment, or the environment in which they are prepared (e.g. cell culture) when such preparation is by recombinant DNA technology practiced in vitro or in vivo. Peptides and nucleic acid can be formulated with diluents or adjuvants and still for practical purposes be isolated—for example the peptides will normally be mixed with gelatin or other carriers if used to coat microtitre plates for use in immunoassays, or will be mixed with pharmaceutically acceptable carriers or diluents when used in diagnosis or therapy.

A “recombinant” peptide refers to a peptide produced via recombinant DNA technology. Recombinantly produced peptides expressed in host cells are considered isolated for the purpose of the present disclosure, as are native or recombinant polypeptides which have been separated, fractionated, or partially or substantially purified by any suitable technique.

The terms “fragment,” “analog,” “derivative,” or “variant” when referring to a GLP-1/glucagon agonist peptide include any peptide which retains at least some desirable activity, e.g., binding to glucagon and/or GLP-1 receptors. Fragments of GLP-1/glucagon agonist peptides provided herein include proteolytic fragments, deletion fragments which exhibit desirable properties during expression, purification, and or administration to a subject.

The term “variant,” as used herein, refers to a peptide that differs from the recited peptide due to amino acid substitutions, deletions, insertions, and/or modifications. Variants can be produced using art-known mutagenesis techniques. Variants can also, or alternatively, contain other modifications—for example a peptide can be conjugated or coupled, e.g., fused to a heterologous amino acid sequence or other moiety, e.g., for increasing half-life, solubility, or stability. Examples of moieties to be conjugated or coupled to a peptide provided herein include, but are not limited to, albumin, an immunoglobulin Fc region, polyethylene glycol (PEG), and the like. The peptide can also be conjugated or produced coupled to a linker or other sequence for ease of synthesis, purification or identification of the peptide (e.g., 6-His), or to enhance binding of the polypeptide to a solid support.

The terms “composition” or “pharmaceutical composition” refer to compositions containing a GLP-1/glucagon agonist peptide provided herein, along with e.g., pharmaceutically acceptable carriers, excipients, or diluents for administration to a subject in need of treatment, e.g., a human subject with T2DM and renal impairment.

The term “pharmaceutically acceptable” refers to compositions that are, within the scope of sound medical judgment, suitable for contact with the tissues of human beings and animals without excessive toxicity or other complications commensurate with a reasonable benefit/risk ratio.

An “effective amount” is that amount of an agent provided herein (e.g., a GLP-1/glucagon agonist peptide such as Cotadutide), the administration of which to a subject, either in a single dose or as part of a series, is effective for treatment, e.g., for improved glycemic control, weight loss, and/or treatment of T2DM in subjects with renal impairment.

As used herein, the terms “subject” and “patient” are used interchangeably. The subject can be an animal. In some aspects of the present disclosure, the subject is a mammal such as a non-human animal (e.g., cow, pig, horse, cat, dog, rat, mouse, monkey or other primate, etc.). In some aspects of the present disclosure, the subject is a cynomolgus monkey. In some aspects of the present disclosure, the subject is a human.

As used herein, a “subject in need thereof” or a “patient in need thereof” refers to an individual for whom it is desirable to treat, e.g., a subject in need of improved glycemic control, weight loss, and/or treatment of T2DM in subjects with renal impairment.

Terms such as “treating” or “treatment” or “to treat” refer to therapeutic measures that cure and/or halt progression of a diagnosed pathologic condition or disorder. Terms such as “preventing” refer to prophylactic or preventative measures that prevent and/or slow the development of a targeted pathologic condition or disorder. Thus, those in need of treatment include those already with the disease or condition. Those in need of prevention include those prone to have the disease or condition and those in whom the disease or condition is to be prevented.

Terms such as “decreasing the severity” refer to therapeutic measures that slow down or lessen the symptoms of a diagnosed pathologic condition or disorder.

As used herein a “GLP-1/glucagon agonist peptide” is a chimeric peptide that exhibits activity at the glucagon receptor of at least about 1%, 5%, 10%, 20%, 30%, 40%, 50%, 60%, 70%, 80%, 90%, 95%, or more relative to native glucagon and also exhibits activity at the GLP-1 receptor of about at least about 1%, 5%, 10%, 20%, 30%, 40%, 50%, 60%, 70%, 80%, 90%, 95%, or more relative to native GLP-1, under the conditions of assay 1.

As used herein the term “native glucagon” refers to naturally-occurring glucagon, e.g., human glucagon, comprising the sequence of SEQ ID NO:1. The term “native GLP-1” refers to naturally-occurring GLP-1, e.g., human GLP-1, and is a generic term that encompasses, e.g., GLP-1(7-36) amide (SEQ ID NO:2), GLP-1(7-37) acid (SEQ ID NO:3), or a mixture of those two compounds. As used herein, a general reference to “glucagon” or “GLP-1” in the absence of any further designation is intended to mean native human glucagon or native human GLP-1, respectively. Unless otherwise indicated, “glucagon” refers to human glucagon, and “GLP-1” refers to human GLP-1.

### II. GLP-1/Glucagon Agonist Peptides

Provided herein are peptides which bind both to a glucagon receptor and to a GLP-1 receptor. Exemplary peptides such as Cotadutide (G933; MEDI0382) are provided in WO 2014/091316 and WO 2017/153575, each of which is herein incorporated by reference in its entirety. In some aspects provided herein, the peptide is Cotadutide, i.e., a 30 amino acid linear peptide with the sequence of HSQGTFTSDX₁₀SEYLDSERARDFVAWLEAGG-acid, wherein X₁₀=lysine with a palmitoyl group conjugated to the epsilon nitrogen, through a gamma glutamic acid linker (i.e., K(gE-palm)) (SEQ ID NO:4) (see FIG. 1.). In some aspects, the peptides provided herein are co-agonists of glucagon and GLP-1 activity. Such peptides are referred to herein as GLP-1/glucagon agonist peptides. GLP-1/glucagon agonist peptides as provided herein possess GLP-1 and glucagon activities with favorable ratios to promote weight loss, prevent weight gain, or to maintain a desirable body weight, and possess optimized solubility, formulatability, and stability. In some aspects, GLP-1/glucagon agonist peptides as provided herein are active at the human GLP1 and human glucagon receptors. In some aspects, GLP-1/glucagon agonist peptides as disclosed have desirable potencies at the glucagon and GLP-1 receptors, and have desirable relative potencies for promoting weight loss.

Cotadutide has a glutamate residue at position 12, and maintains robust activity at both the glucagon and GLP-1 receptors. The corresponding residue is lysine in exendin-4 (exenatide) and glucagon and is serine in GLP-1. Although this residue is not thought to contact the receptor, changes in charge from positive to negative may modify the adjacent environment. Furthermore, Cotadutide has a glutamate residue at position 27. Residue 27 is Lysine in exendin 4 and is an uncharged hydrophobic residue in GLP1 (valine) and glucagon (methionine). The lysine of exendin 4 makes electrostatic interactions with the GLP1 receptor at residues Glu127 and Glu24 (C. R. Underwood et al J Biol Chem 285 723-730 (2010); S. Runge et al J Biol Chem 283 11340-11347 (2008)). While a loss of GLP1R potency might be expected when the charge at position 27 is changed to negative, the change is compatible with GLP1R activity in Cotadutide.

Cotadutide is palmitoylated to extend its half-life by association with serum albumin, thus reducing its propensity for renal clearance.

Alternatively or in addition, a GLP-1/glucagon agonist peptide as disclosed herein can be associated with a heterologous moiety, e.g., to extend half-life. The heterologous moiety can be a protein, a peptide, a protein domain, a linker, an organic polymer, an inorganic polymer, a polyethylene glycol (PEG), biotin, an albumin, a human serum albumin (HSA), a HSA FcRn binding portion, an antibody, a domain of an antibody, an antibody fragment, a single chain antibody, a domain antibody, an albumin binding domain, an enzyme, a ligand, a receptor, a binding peptide, a non-FnIII scaffold, an epitope tag, a recombinant polypeptide polymer, a cytokine, and a combination of two or more of such moieties.

Cotadutide can be administered in a titrated dose, e.g., at an initial dose, then at a second higher dose, and optionally at a third higher dose thereafter. The initial dose, and optionally the second dose, can be administered for about 7 days to about 28 days (e.g., about 7 days to about 14 days). The initial dose can be at least 20 μg daily and administered. The highest dose (e.g., the second dose or the third dose) can be a dose that does not exceed 600 μg daily. The highest dose (e.g., the second dose or the third dose) can be a dose that does not exceed 300 μg daily.

### III. Methods of Making GLP-1/Glucagon Agonist Peptides

GLP-1/glucagon agonist peptides for uses provided herein can be made by any suitable method. For example, in some aspects provided herein, the GLP-1/glucagon agonist peptides for uses provided herein are chemically synthesized by methods well known to those of ordinary skill in the art, e.g., by solid phase synthesis as described by Merrifield (1963, J. Am. Chem. Soc. 85:2149-2154). Solid phase peptide synthesis can be accomplished, e.g., by using automated synthesizers, using standard reagents, e.g., as explained in Example 1 of WO 2014/091316, which is herein incorporated by reference in its entirety.

Alternatively, GLP-1/glucagon agonist peptides for uses provided herein can be produced recombinantly using a convenient vector/host cell combination as would be well known to the person of ordinary skill in the art. A variety of methods are available for recombinantly producing GLP-1/glucagon agonist peptides. Generally, a polynucleotide sequence encoding the GLP-1/glucagon agonist peptide is inserted into an appropriate expression vehicle, e.g., a vector which contains the necessary elements for the transcription and translation of the inserted coding sequence. The nucleic acid encoding the GLP-1/glucagon agonist peptide is inserted into the vector in proper reading frame. The expression vector is then transfected into a suitable host cell which will express the GLP-1/glucagon agonist peptide. Suitable host cells include without limitation bacteria, yeast, or mammalian cells. A variety of commercially-available host-expression vector systems can be utilized to express the GLP-1/glucagon agonist peptides described herein.

### VI. Pharmaceutical Compositions and Kits

As provided herein, GLP-1/glucagon agonist peptides (e.g., Cotadutide) can be used to improve glycemic control, reduce weight, decrease urine albumin:creatinine ratio (UACR) and/or treat diabetic kidney disease (DKD) in a human patient with DKD or renal insufficiency and type 2 diabetes mellitus (T2DM). As provided herein, GLP-1/glucagon agonist peptides (e.g., Cotadutide) can be used to improve glycemic control, reduce weight, decrease urine albumin:creatinine ratio (UACR) and/or treat chronic kidney disease (CKD) in a human patient with CKD.

A pharmaceutical composition comprising a GLP-1/glucagon agonist peptide (e.g., Cotadutide) can be formulated for injection. A pharmaceutical composition comprising a GLP-1/glucagon agonist peptide (e.g., Cotadutide) can be formulated for subcutaneous administration.

A pharmaceutical composition comprising a GLP-1/glucagon agonist peptide (e.g., Cotadutide) can comprise about 50 μg, about 100 μg, about 200 μg, about 300 μg, or about 600 μg of the GLP-1/glucagon agonist peptide (e.g., Cotadutide).

A pharmaceutical composition comprising a GLP-1/glucagon agonist peptide (e.g., Cotadutide) can be provided in an injection pen device. The injection pen device can be for subcutaneous administration.

### VII. Methods of Use

As provided herein, GLP-1/glucagon agonist peptides (e.g., Cotadutide) can be used to improve glycemic control, reduce weight, decrease urine albumin:creatinine ratio (UACR) and/or treat diabetic kidney disease (DKD) in a human patient with DKD or renal insufficiency and type 2 diabetes mellitus (T2DM). As provided herein, GLP-1/glucagon agonist peptides (e.g., Cotadutide) can be used to improve glycemic control, reduce weight, decrease urine albumin:creatinine ratio (UACR) and/or treat chronic kidney disease (CKD) in a human patient with CKD.

In some aspects, a patient with renal impairment has an eGFR of ≥30 and <60 mL/min/1.73 m². In some aspects, a patient with DKD has an eGFR of ≥20 to ≤90 mL/min/1.73 m². A patient's eGFR can determined using the chronic kidney disease epidemiology collaboration equation (CKD-EPI).

As provided herein a method of improving glycemic control in a human subject with T2DM and DKD or T2DM and renal insufficiency can comprise administering to the subject a GLP-1/glucagon agonist peptide (e.g., Cotadutide). As provided herein a method of improving glycemic control in a human subject with CKD can comprise administering to the subject a GLP-1/glucagon agonist peptide (e.g., Cotadutide). This disclosure also provides a GLP-1/glucagon agonist peptide (e.g., Cotadutide) for improving glycemic control in a human subject with DKD or renal insufficiency T2DM. This disclosure also provides a GLP-1/glucagon agonist peptide (e.g., Cotadutide) for improving glycemic control in a human subject with CKD. This disclosure also provides a GLP-1/glucagon agonist peptide (e.g., Cotadutide) for use in the manufacture of a medicament for improving glycemic control in a human subject with T2DM and DKD or T2DM and renal insufficiency. This disclosure also provides a GLP-1/glucagon agonist peptide (e.g., Cotadutide) for use in the manufacture of a medicament for improving glycemic control in a human subject with CKD. The GLP-1/glucagon agonist peptide (e.g., Cotadutide) for improving glycemic control can be administered or for administration at a dose of 20-600 50-600 50-300 or 100-300 optionally wherein the administration is by injection (e.g., subcutaneous administration). The GLP-1/glucagon agonist peptide (e.g., Cotadutide) for improving glycemic control can be administered or for administration in titrated doses, e.g., at an initial dose of 100 μg, then a second dose of 200 then a third dose of 300 μg. The initial dose can be administered for about 7 days. The second dose can be administered for about 7 days. The administration can be an adjunct to diet and exercise.

As provided herein a method of reducing weight in a human subject with T2DM and DKD or T2DM and renal insufficiency can comprise administering to the subject a GLP-1/glucagon agonist peptide (e.g., Cotadutide). As provided herein a method of reducing weight in a human subject with CKD can comprise administering to the subject a GLP-1/glucagon agonist peptide (e.g., Cotadutide). This disclosure also provides a GLP-1/glucagon agonist peptide (e.g., Cotadutide), for reducing weight in a human subject with T2DM and DKD or T2DM and renal insufficiency. This disclosure also provides a GLP-1/glucagon agonist peptide (e.g., Cotadutide), for reducing weight in a human subject with CKD. This disclosure also provides a GLP-1/glucagon agonist peptide (e.g., Cotadutide), for use in the manufacture of a medicament for reducing weight in a human subject with T2DM and DKD or T2DM and renal insufficiency. This disclosure also provides a GLP-1/glucagon agonist peptide (e.g., Cotadutide), for use in the manufacture of a medicament for reducing weight in a human subject with CKD. In some aspects, a patient's weight is reduced by e.g., at least 5% or by at least 10%. In some aspects, a patient's weight is reduced by about 5% to about 40%. The GLP-1/glucagon agonist peptide (e.g., Cotadutide) for reducing weight can be administered or for administration at a dose of 20-600 μg, 50-600 μg, 50-300 μg, or 100-300 μg, optionally wherein the administration is by injection (e.g., subcutaneous administration). The GLP-1/glucagon agonist peptide (e.g., Cotadutide) for reducing weight can be administered or for administration in titrated doses, e.g., at an initial dose of 100 then a second dose of 200 then a third dose of 300 μg. The initial dose can be administered for about 7 days. The second dose can be administered for about 7 days. The administration can be an adjunct to diet and exercise.

As provided herein a method of treating DKD in a human subject with T2DM and DKD can comprise administering to the subject a GLP-1/glucagon agonist peptide (e.g., Cotadutide). This disclosure also provides a GLP-1/glucagon agonist peptide (e.g., Cotadutide) for treating DKD in a human subject with T2DM and DKD. This disclosure also provides a GLP-1/glucagon agonist peptide (e.g., Cotadutide) for use in the manufacture of a medicament for treating DKD in a human subject with T2DM and DKD. The GLP-1/glucagon agonist peptide (e.g., Cotadutide) for treating DKD can be administered or for administration at a dose of 20-600 50-600 50-300 or 100-300 optionally wherein the administration is by injection (e.g., subcutaneous administration). The GLP-1/glucagon agonist peptide (e.g., Cotadutide) for treating DKD can be administered or for administration in titrated doses, e.g., at an initial dose of 100 μg, then a second dose of 200 μg, then a third dose of 300 μg. The initial dose can be administered for about 7 days. The second dose can be administered for about 7 days. The administration can be an adjunct to diet and exercise.

As provided herein a method of treating CKD in a human subject with CKD can comprise administering to the subject a GLP-1/glucagon agonist peptide (e.g., Cotadutide). This disclosure also provides a GLP-1/glucagon agonist peptide (e.g., Cotadutide) for treating CKD in a human subject with CKD. This disclosure also provides a GLP-1/glucagon agonist peptide (e.g., Cotadutide) for use in the manufacture of a medicament for treating CKD in a human subject with CKD. The GLP-1/glucagon agonist peptide (e.g., Cotadutide) for treating CKD can be administered or for administration at a dose of 20-600 μg, 50-600 μg, 50-300 μg, or 100-300 μg, optionally wherein the administration is by injection (e.g., subcutaneous administration). The GLP-1/glucagon agonist peptide (e.g., Cotadutide) for treating CKD can be administered or for administration in titrated doses, e.g., at an initial dose of 100 μg, then a second dose of 200 μg, then a third dose of 300 μg. The initial dose can be administered for about 7 days. The second dose can be administered for about 7 days. The administration can be an adjunct to diet and exercise.

As provided herein a method of decreasing urine albumin:creatinine ratio (UACR) in a human subject with T2DM and DKD or T2DM and renal insufficiency can comprise administering to the subject a GLP-1/glucagon agonist peptide (e.g., Cotadutide). As provided herein a method of decreasing urine albumin:creatinine ratio (UACR) in a human subject with CKD can comprise administering to the subject a GLP-1/glucagon agonist peptide (e.g., Cotadutide). This disclosure also provides a GLP-1/glucagon agonist peptide (e.g., Cotadutide) for decreasing UACR in a human subject with T2DM and DKD. This disclosure also provides a GLP-1/glucagon agonist peptide (e.g., Cotadutide) for decreasing UACR in a human subject with CKD. This disclosure also provides a GLP-1/glucagon agonist peptide (e.g., Cotadutide) for use in the manufacture of a medicament for decreasing UACR in a human subject with T2DM and DKD or T2DM and renal insufficiency. This disclosure also provides a GLP-1/glucagon agonist peptide (e.g., Cotadutide) for use in the manufacture of a medicament for decreasing UACR in a human subject with CKD. In certain aspects, the GLP-1/glucagon agonist peptide (e.g., Cotadutide) is more effective in decreasing UACR than semaglutide. The GLP-1/glucagon agonist peptide (e.g., Cotadutide) for decreasing UACR can be administered or for administration at a dose of 20-600 μg, 50-600 μg, 50-300 μg, or 100-300 μg, optionally wherein the administration is by injection (e.g., subcutaneous administration). The GLP-1/glucagon agonist peptide (e.g., Cotadutide) for decreasing UACR can be administered or for administration in titrated doses, e.g., at an initial dose of 100 μg, then a second dose of 200 μg, then a third dose of 300 μg. The initial dose can be administered for about 7 days. The second dose can be administered for about 7 days. The administration can be an adjunct to diet and exercise.

In some aspects provided herein, the GLP-1/glucagon agonist peptide (e.g., Cotadutide) can reduce the mixed-meal tolerance test (MMTT) plasma glucose area under the curve (AUC)_{0-4 hours} in a patient. The reduction can occur, e.g., within 2 weeks, within 12 weeks, within 14 weeks, or within 26 weeks from the first administration of the GLP-1/glucagon agonist peptide (e.g., Cotadutide).

In some aspects provided herein, the GLP-1/glucagon agonist peptide (e.g., Cotadutide) can reduce continuous glucose monitoring (CGM) glucose AUC₀₋₂₄ in a patient. The reduction can occur, e.g., within 2 weeks, within 12 weeks, within 14 weeks, or within 26 weeks from the first administration of the GLP-1/glucagon agonist peptide (e.g., Cotadutide).

In some aspects provided herein, the GLP-1/glucagon agonist peptide (e.g., Cotadutide) can reduce fasting plasma glucose (FPG) in a patient. The reduction can occur, e.g., within 2 weeks, within 12 weeks, within 14 weeks, or within 26 weeks from the first administration of the GLP-1/glucagon agonist peptide (e.g., Cotadutide).

In some aspects provided herein, the GLP-1/glucagon agonist peptide (e.g., Cotadutide) can reduce Hemoglobin A1c (HbA1c) in a patient. The reduction can occur, e.g., within 2 weeks, within 12 weeks, within 14 weeks, or within 26 weeks from the first administration of the GLP-1/glucagon agonist peptide (e.g., Cotadutide).

In some aspects provided herein, the GLP-1/glucagon agonist peptide (e.g., Cotadutide) can reduce body weight of a patient, e.g., by at least 5% or by at least 10%. The reduction can occur, e.g., within 2 weeks, within 12 weeks, within 14 weeks, or within 26 weeks from the first administration of the GLP-1/glucagon agonist peptide (e.g., Cotadutide).

In some aspects provided herein, the GLP-1/glucagon agonist peptide (e.g., Cotadutide) can produce euglycemic glucose levels in a patient. In some aspects provided herein, the GLP-1/glucagon agonist peptide (e.g., Cotadutide) can prevent hyperglycemic glucose levels in the patient. In some aspects provided herein, the GLP-1/glucagon agonist peptide (e.g., Cotadutide) can reduce the frequency and/or length of hyperglycemia in the patient.

In some aspects provided herein, the GLP-1/glucagon agonist peptide (e.g., Cotadutide) can improve insulin resistance in a patient. Insulin resistance can be measured, for example, using the Homeostatic Model Assessment of Insulin Resistance (HOMA-IR) and/or the MATSUDA index. The HOMA-IR is explained, for example, in Matthews D R, et al., Diabetologia 28: 412-419 (1985), which is herein incorporated by reference in its entirety. The MATSUDA index is explained, for example, in Matsuda M, and DeFronzo R A, Diabetes Care 22:1462-1470 (1999), which is herein incorporated by reference in its entirety.

In some aspects provided herein, the GLP-1/glucagon agonist peptide (e.g., Cotadutide) can improve beta cell function in a patient.

As provided herein, the GLP-1/glucagon agonist peptide (e.g., Cotadutide) can be titrated. The titration can comprise an initial dose and a second dose, wherein the second dose is higher than the initial dose. The titration can further comprise a third dose, wherein the third dose is higher than the second dose. The titration can further comprise a fourth dose, wherein the fourth dose is higher than the third dose. The titration can further comprise a fifth dose, wherein the firth dose is higher than the fourth dose. The titration can further comprise a sixth dose, wherein the sixth dose is higher than the fifth dose. In some aspects, the maximum dose of the GLP-1/glucagon agonist peptide (e.g., Cotadutide) does not exceed 300 μg. In some aspects, the maximum dose of the GLP-1/glucagon agonist peptide (e.g., Cotadutide) does not exceed 600 μg.

In certain aspects, the initial dose is administered for 1 day to about 2 weeks (e.g., for about 4 days, for about 1 week, or for about 2 weeks. In certain aspects, the second, third, fourth, fifth, and/or sixth dose is administered for about 1 week to about 2 weeks.

As provided herein, the GLP-1/glucagon agonist peptide (e.g., Cotadutide) can be titrated from 50 μg to 300 μg. For example, the GLP-1/glucagon agonist peptide (e.g., Cotadutide) can be administered at 50 μg daily for 1 day to about 2 weeks (e.g., for about 4 days, for about 1 week, or for about 2 weeks), then at 100 μg daily for about 1 week to about 2 weeks, then at 200 μg daily for about 1 week to about 2 weeks, then at 300 μg daily.

As provided herein, the GLP-1/glucagon agonist peptide (e.g., Cotadutide) can be titrated from 100 μg to 300 μg. For example, the GLP-1/glucagon agonist peptide (e.g., Cotadutide) can be administered at 100 μg daily for 1 day to about 2 weeks (e.g., for about 4 days, for about 1 week, or for about 2 weeks), then at 200 μg daily for about 1 week to about 2 weeks, then at 300 μg daily.

As provided herein, the GLP-1/glucagon agonist peptide (e.g., Cotadutide) can be titrated from 50 μg to 600 μg. For example, the GLP-1/glucagon agonist peptide (e.g., Cotadutide) can be administered at 50 μg daily for 1 day to about 2 weeks (e.g., for about 4 days, for about 1 week, or for about 2 weeks), then at 100 μg daily for about 1 week to about 2 weeks, then at 200 μg daily for about 1 week to about 2 weeks, then at 400 μg daily for about 1 week to about 2 weeks, then at 600 μg daily.

In some aspects, the GLP-1/glucagon agonist peptide (e.g., Cotadutide) is administered for at least two weeks, for at least 12 weeks, for at least 14 weeks, or for at least 26 weeks. In some aspects, the GLP-1/glucagon agonist peptide (e.g., Cotadutide) is administered at the maximum dose for at least one week or for at least two weeks.

In some aspects, the GLP-1/glucagon agonist peptide (e.g., Cotadutide) is administered or is for administration subcutaneously, optionally via an in an injection pen device.

As provided herein, the human subject discussed in any of the aspects provided herein can have type 2 diabetes mellitus (T2DM) and diabetic kidney disease (DKD) or can have T2DM and renal insufficiency.

As provided herein, the human subject discussed in any aspects provided herein can have CKD. The human subject with CKD can be a subject with diabetes, e.g., T2DM. The human subject with CKD can be a subject without diabetes.

As provided herein, the human subject discussed in any of the aspects provided herein can have a body mass index (BMI) of at least 23 kg/m² or at least 25 kg/m². As provided herein, the human subject discussed in any of the aspects provided herein can have a BMI of no more than 40 kg/m². As provided herein, the human subject discussed in any of the aspects provided herein can have a BMI of 23 kg/m² to 40 kg/m². As provided herein, the human subject discussed in any of the aspects provided herein can have a BMI of 25 kg/m² to 40 kg/m².

As provided herein, the human subject discussed in any of the aspects provided herein can have an hemoglobin A1c (HbA1c) of <8.0%

As provided herein, the human subject discussed in any of the aspects provided herein can have an estimated glomerular filtration rate (eGFR) of less than 90 mL/min/1.73 m² prior to the administration or less than 60 mL/min/1.73 m² prior to the administration. As provided herein, the human subject discussed in any of the aspects provided herein can have an eGFR of at least 20 mL/min/m² prior to the administration or at least 30 mL/min/m² prior to the administration. As provided herein, the human subject discussed in any of the aspects provided herein can have an eGFR of at least 20 mL/min/m² and at least 20 mL/min/m² As provided herein, the human subject discussed in any of the aspects provided herein can have an eGFR of at least 30 30 mL/min/m² and less than 60 mL/min/1.73 m² prior to the administration.

In some aspects, a human subject provided herein is receiving treatment with insulin prior to the administration of the GLP-1/glucagon agonist peptide (e.g., Cotadutide). In some aspects provided herein, the GLP-1/glucagon agonist peptide (e.g., Cotadutide) can reduce insulin use by a patient.

In some aspects, a human subject provided herein is receiving treatment with metformin prior to the administration of the GLP-1/glucagon agonist peptide (e.g., Cotadutide). In some aspects, a human subject provided herein is receiving treatment with an SGLT2 inhibitor prior to the administration of the GLP-1/glucagon agonist peptide (e.g., Cotadutide). In some aspects, a human subject provided herein is receiving treatment with insulin, metformin, and/or an SGLT2 inhibitor prior to the administration of the GLP-1/glucagon agonist peptide (e.g., Cotadutide).

### EXAMPLES

### Example 1: Cotadutide Improves Insulin Sensitivity and Restores Normal Insulin Secretory Capacity in Diet-Induced Obese Mice

Hyperinsulinemic clamps were used in diet induced obese (DIO) mice to determine whether Cotadutide improves insulin sensitivity and β-cell function. Following 28 days of daily dosing with Cotadutide (10 nmol/kg), Liraglutide (5 nmol/kg; GLP-1 agonist) or g1437 (5 nmol/kg; a Gcg analog), mice underwent continuous infusion of 4 mU/kg/min insulin and [3H]-glucose to assess glucose turnover. Tissue-specific glucose uptake was assessed using 14C-2-deoxyglucose.

Lower body weight was observed in all groups compared with vehicle control, with g1437 eliciting the largest response, and Liraglutide the least. Fasting glucose was significantly higher in g1437 treated mice. Fasting insulin was dramatically lower in Cotadutide and g1437 groups compared with vehicle. Despite an equal rate of human insulin infusion between groups, the total insulin levels during the clamp remained lower in Cotadutide and g1437 treated mice. The glucose infusion rate, to maintain euglycemia ˜130 mg/dL, was significantly higher in Cotadutide treated mice in line with increased glucose disposal rate. Hepatic glucose production was suppressed in all groups, despite significantly lower levels of insulin in Cotadutide and g1437 treated mice. Finally, glucose uptake was elevated in brown adipose tissue in Cotadutide treated mice, which was confirmed in a separate study of [¹⁸F]FDG uptake by PET imaging.

These experiments demonstrate that Cotadutide improves insulin sensitivity in concert with dramatic reductions for insulin demand, which can result in recovery of endogenous β-cell function in Type 2 Diabetes Mellitus (T2DM).

### Example 2: Clinical Evaluation of Cotadutide in Patients with T2DM and Renal Impairment (Phase 2a)

A Phase 2a randomized, placebo-controlled, double-bind study was performed to demonstrate the efficacy and safety of Cotadutide in subjects with Type 2 Diabetes Mellitus (T2DM) with renal impairment.

### (A) Subjects

A total of 101 subjects consented to participate in the study. The study enrolled subjects with T2DM and renal impairment. The subjects were screened for the following inclusion and exclusion criteria.

Inclusion criteria:
Male and female subjects, aged ≥18 years and <85 years at screening;
Body mass index (BMI) between 25 kg/m² and 40 kg/m² (inclusive) at screening;
HbA1c range of 6.5% to 10.5% (inclusive) at the time of screening;
Diagnosed with T2DM with glucose control managed with any insulin and/or oral therapy combination where no significant dose changes of oral therapy of more than 50% had occurred in the 3 months prior to screening (at least 50% of the subject N=20) enrolled were required to have been taking insulin at a total daily dose of ≥20 units);
Renal impairment with eGFR ≥30 and <60 mL/min/1.73 m² at screening. Approximately 16 subjects (40%) were required to have a screening eGFR ≥45 and <60 mL/min/1.73 m² (in order to ensure subjects at both ends of the moderate renal impairment spectrum were adequately represented). eGFR was determined by the chronic kidney disease-epidemiology collaboration (CKD-EPI) equation. (eGFR=141×min(S_{Cr}/κ, 1)^{α}×max (S_{Cr}/κ, 1)⁻¹²⁰⁹×0.993^{Age}×1.018 [if female]×1.159 [if Black]; wherein S_{Cr} (standardized serum creatinine)=mg/dL, κ=0.7 (females) or 0.9 (males), α=−0.329 (females) or −0.411 (males), min=indicates the minimum of SCr/κ or 1, max=indicates the maximum of SCr/κ or 1, and age=years)

Exclusion Criteria:
History or presence of significant medical or psychological conditions;
Any subject who had received another investigational product as part of a clinical study or a GLP-1 analogue-containing preparation within the last 30 days or 5 half-lives of the drug (if known; whichever was longer) at the time of Visit 2;
Any subject who had received any of the following medications within the specified timeframe prior to the start of the study (Visit 2):
   Herbal preparations within 1 week prior to the start of dosing (Visit 4) or drugs licensed for control of body weight or appetite (e.g., orlistat, bupropion-naltrexone, phentermine-topiramate, phentermine, or lorcaserin) within 30 days (or 5 half-lives of the drug) prior to the start of dosing (Visit 4);
   Aspirin (acetylsalicylic acid) at a dose greater than 150 mg once daily and within the last 3 days prior to the start of the run-in period (Visit 2);
   Paracetamol (acetaminophen) or paracetamol-containing preparations at a total daily dose of greater than 3000 mg and within the last 3 days prior to the start of the run-in period (Visit 2);
   Ascorbic acid (vitamin C) supplements at a total daily dose of greater than 1000 mg and within the last 3 days prior to the start of the run-in period (Visit 2); or
   Opiates, domperidone, metoclopramide, or other drugs known to alter gastric emptying and within 2 weeks prior to the start of dosing (Visit 4).
Symptoms of acutely decompensated blood glucose control (e.g., thirst, polyuria, or weight loss), a history of type 1 diabetes mellitus, or diabetic ketoacidosis;
Subjects who had undergone a renal transplant;
Subjects with suspicion of acute or subacute renal function deterioration (e.g., subjects with large fluctuations of creatinine values documented within the 6 months prior to screening;
Significant inflammatory bowel disease, gastroparesis, or other severe disease or surgery affecting the upper gastrointestinal (GI) tract (including weight-reducing surgery and procedures) that could have affected gastric emptying or could have affected the interpretation of safety and tolerability data;
History of acute or chronic pancreatitis;
Significant hepatic disease (except for non-alcoholic steatohepatitis or non-alcoholic fatty liver disease without portal hypertension or cirrhosis) and/or subjects with any of the following results:
   Aspartate transaminase (AST) ≥3×upper limit of normal (ULN);
   Alanine transaminase (ALT) ≥3×ULN; or
   Total bilirubin ≥2×ULN
Poorly controlled hypertension defined as (a) Systolic BP >180 mm Hg (b) Diastolic BP ≥100 mm Hg after 10 minutes of seated rest and confirmed by repeated measurement at screening. Subjects who failed BP screening criteria may have been considered for 24-hour ambulatory blood pressure monitoring (ABPM). Subjects who maintained a mean 24-hour systolic BP≤180 or diastolic BP<100 mm Hg with a preserved nocturnal dip of >15% were considered eligible;
Unstable angina pectoris, myocardial infarction, transient ischemic attack, or stroke within 3 months prior to screening, or subjects who had undergone percutaneous coronary intervention or a coronary artery bypass graft within the past 6 months or who were due to undergo these procedures at the time of screening;
Severe congestive heart failure (New York Heart Association Class III or IV);
Basal calcitonin level >50 ng/L at screening or history/family history of medullary thyroid carcinoma or multiple endocrine neoplasia;
History of neoplastic disease within 5 years prior to screening, except for adequately treated basal cell skin cancer, squamous cell skin cancer, or in situ cervical cancer;
Any positive results for serum hepatitis B surface antigen (HBsAg), hepatitis C antibody, or human immunodeficiency virus (HIV) antibody;
Nephrotic range proteinuria defined as spot urine albumin:creatinine ratio (UACR) >250 mg/mmol at screening;
History of substance dependence, alcohol abuse, or excessive alcohol intake.

Of the 101 subjects who consented to participate, 41 subjects were considered screen failures, and 41 subjects were randomized (21 subjects to the Cotadutide group and 20 subjects to the placebo group). Of the 41 randomized subjects, 28 were on ≥20 U/day insulin, while 2 were on <20 U/day insulin and/or oral treatment (1 each in the placebo and Cotadutide groups), and 11 were on oral treatment only (5 and 6 subjects in the placebo and Cotadutide groups, respectively) for management of glycemic control.

A majority completed treatment as planned. Four subjects (9.8%, 1 subject treated with placebo and 3 subjects treated with Cotadutide) discontinued placebo/Cotadutide and did not complete treatment: 3 subjects (7.3%) due to an adverse event (AE; 1 subject treated with placebo and 2 subjects treated with Cotadutide) and 1 subject (2.4%) due to death (treated with Cotadutide). All subjects, except for the subject who died, completed the study.

The intent-to-treat (ITT) population was defined as all subjects that were randomized and received any amount of Cotadutide or placebo, analyzed according to randomized treatment assignment. All efficacy analyses were performed on the ITT population unless otherwise specified.

The as-treated population was defined as all subjects who received at least 1 dose of Cotadutide or placebo and were analyzed according to the treatment actually received. All safety analyses were performed on the as-treated population.

All 41 subjects who received Cotadutide or placebo were included in the ITT and As-treated populations.

The pharmacokinetic (PK) population included all subjects who received at least 1 dose of Cotadutide or placebo and had at least one PK sample taken with a value above the lower limit of quantitation. All 21 subjects who received Cotadutide had evaluable postdose PK data and were included in the PK population.

The immunogenicity population included all subjects in the as-treated population who had at least one serum immunogenicity result.

The demographics of the Cotadutide group and Placebo group were balanced (Table 1).

TABLE 1
Demographics

| | | | | | | |
|---|---|---|---|---|---|---|
| Placebo | MEDI0382 | Placebo | MEDI0382 | | | |
| (Insulin | (Insulin | (Insulin | (Insulin | | | |
| Dose ≥ 20 | Dose 20 | Dose < 20 | Dose < 20 | Placebo | MEDI0382 | |
| U/day) | U/day) | U/day^{a}) | U/day^{a}) | (Total) | (Total) | Total |
| N = 14 | N = 14 | N = 6 | N = 7 | N = 20 | N = 21 | N = 41 |

Age (years)

| | | | | | | | |
|---|---|---|---|---|---|---|---|
| N | 14 | 14 | 6 | 7 | 20 | 21 | 41 |
| Mean | 70.6 | 70.7 | 71.3 | 71.9 | 70.9 | 71.1 | 71.0 |
| (SD) | (3.9) | (6.6) | (6.6) | (9.2) | (4.7) | (7.4) | (6.1) |
| Median | 70.0 | 71.5 | 72.0 | 71.0 | 70.5 | 71.0 | 71.0 |
| (Min, Max) | (65, 79) | (59, 81) | (63, 81) | (57, 83) | (63, 81) | (57, 83) | (57, 83) |

Sex

| | | | | | | | |
|---|---|---|---|---|---|---|---|
| N | 14 | 14 | 6 | 7 | 20 | 21 | 41 |
| Female | 7 | 6 | 4 | 3 | 11 | 9 | 20 |
| | (50.0%) | (42.9%) | (66.7%) | (42.9%) | (55.0%) | (42.9%) | (48.8%) |
| Male | 7 | 8 | 2 | 4 | 9 | 12 | 21 |
| | (50.0%) | (57.1%) | (33.3%) | (57.1%) | (45.0%) | (57.1%) | (51.2%) |

Weight (kg)

| | | | | | | | |
|---|---|---|---|---|---|---|---|
| N | 14 | 14 | 6 | 7 | 20 | 21 | 41 |
| Mean | 93.09 | 94.06 | 88.20 | 95.90 | 91.63 | 94.68 | 93.19 |
| (SD) | (17.57) | (9.57) | (11.04) | (28.73) | (15.77) | (17.55) | (16.57) |
| Median | 90.20 | 97.30 | 83.75 | 94.00 | 89.15 | 97.30 | 94.00 |
| (Min, Max) | (56.9, 120.5) | (75.1, 105.2) | (78.5, 107.6) | (68.6, 153.8) | (56.9, 120.5) | (68.6, 153.8) | (56.9, 153.8) |

Height (cm)

| | | | | | | | |
|---|---|---|---|---|---|---|---|
| N | 14 | 14 | 6 | 7 | 20 | 21 | 41 |
| Mean | 165.62 | 169.89 | 170.17 | 171.71 | 166.99 | 170.50 | 168.79 |
| (SD) | (10.92) | (8.04) | (7.22) | (13.43) | (10.00) | (9.84) | (9.95) |
| Median | 171.00 | 171.50 | 171.00 | 176.00 | 171.00 | 174.00 | 172.00 |
| (Min, Max) | (148.0, 178.0) | (153.0, 179.0) | (158.0, 180.0) | (156.0, 194.0) | (148.0, 180.0) | (153.0, 194.0) | (148.0, 194.0) |

Body mass index (kg/m²)

| | | | | | | | |
|---|---|---|---|---|---|---|---|
| N | 14 | 14 | 6 | 7 | 20 | 21 | 41 |
| Mean | 33.960 | 32.723 | 30.515 | 31.863 | 32.927 | 32.436 | 32.675 |
| (SD) | (5.895) | (3.984) | (3.966) | (4.469) | (5.526) | (4.060) | (4.776) |
| Median | 34.655 | 32.100 | 28.900 | 30.700 | 31.715 | 32.080 | 31.980 |
| (Min, Max) | (25.98, 41.70) | (26.25, 39.30) | (27.38, 38.12) | (26.47, 40.87) | (25.98, 41.70) | (26.25, 40.87) | (25.98, 41.70) |

Max = maximum;
Min = minimum;
SD = standard deviation
^{a}5/6 subjects in the Placebo Insulin Dose < 20 U/day group and 6/7 subjects in the MEDI0382 Insulin Dose < 20 U/day group were only on oral antidiabetic treatment.

Most subjects were White, non-Hispanic or Latino, and had equal representation of males and females. There were no notable differences in mean weight, height, or body mass index (BMI) of the As-treated population between the Cotadutide group and placebo group. Additionally, there were no clinically noteworthy differences in demographics comparing subjects on <20 U/day insulin vs ≥20 U/day insulin.

Baseline mean eGFR level, HbA1c level, fasting glucose level, duration of T2DM, and insulin dose were balanced between the overall Cotadutide group and Placebo group (Table 2).

TABLE 2
Baseline Characteristics

| | | | |
|---|---|---|---|
| Placebo | MEDI0382 | Placebo | MEDI0382 |
| (Insulin | (Insulin | (Insulin | (Insulin |
| Dose ≥ 20 | Dose ≥ 20 | Dose < 20 | Dose < 20 |
| U/day) | U/day) | U/day^{a}) | U/day^{a}) |
| N = 14 | N = 14 | N = 6 | N = 7 |

eGFR (mL/min/1.73 m²)

| | | | | |
|---|---|---|---|---|
| n | 14 | 14 | 6 | 7 |
| Mean | 46.24 | 43.84 | 50.88 | 46.50 |
| (SD) | (8.72) | (9.32) | (8.74) | (7.65) |
| Median | 47.10 | 44.60 | 52.65 | 45.10 |
| (Min, Max) | (30.7, 58.2) | (30.3, 57.7) | (36.4, 59.6) | (38.2, 58.2) |

eGFR (mL/min/1.73 m²)

| | | | | |
|---|---|---|---|---|
| n | 14 | 14 | 6 | 7 |
| 30-44 | 5 | 7 | 1 | 3 |
| | (35.7%) | (50.0%) | (16.7%) | (42.9%) |
| 45-59 | 9 | 7 | 5 | 4 |
| | (64.3%) | (50.0%) | (83.3%) | (57.1%) |

HbA1c (%)

| | | | | |
|---|---|---|---|---|
| n | 14 | 14 | 6 | 7 |
| Mean | 8.23 | 7.89 | 7.07 | 7.77 |
| | (1.36) | (0.76) | (0.45) | (0.75) |
| Median | 7.85 | 7.85 | 7.00 | 7.90 |
| (Min, Max) | (6.6, 10.3) | (6.7, 9.7) | (6.7, 7.9) | (6.5, 8.5) |

Fasting Glucose (mg/dL)

| | | | | |
|---|---|---|---|---|
| n | 14 | 14 | 6 | 7 |
| Mean | 185.2842 | 171.1256 | 160.6783 | 157.5463 |
| (SD) | (54.7642) | (30.5492) | (38.0118) | (15.4352) |
| Median | 179.2990 | 173.4425 | 159.4770 | 154.0710 |
| (Min, Max) | (107.219, 293.726) | (119.833, 233.359) | (115.328, 202.725) | (141.457, 181.101) |

Duration of T2DM (yr)

| | | | | |
|---|---|---|---|---|
| n | 14 | 14 | 6 | 7 |
| Mean | 16.807 | 17.536 | 13.800 | 13.816 |
| (SD) | (7.462) | (8.018) | (6.518) | (9.662) |
| Median | 18.000 | 18.950 | 17.000 | 13.000 |
| (Min, Max) | (3.00, 31.00) | (2.30, 29.00) | (5.00, 19.80) | (3.00, 29.00) |

Insulin Dose (U/day)

| | | | | |
|---|---|---|---|---|
| n | 14 | 14 | 6 | 7 |
| Mean | 81.21 | 75.07 | 2.33 | 1.43 |
| (SD) | (39.24) | (62.88) | (5.72) | (3.78) |
| Median | 70.00 | 56.50 | 0.00 | 0.00 |
| (Min, Max) | (28.0, 167.0) | (18.0, 250.0) | (0.0, 14.0) | (0.0, 10.0) |

Oral Anti-glycemic Medication at Screening

| | | | | |
|---|---|---|---|---|
| n | 6 | 9 | 6 | 7 |
| Yes | 6 | 9 | 6 | 7 |
| | (100%) | (100%) | (100%) | (100%) |
| No | 0 | 0 | 0 | 0 |

| | Placebo | MEDI0382 | |
|---|---|---|---|
| | (Total) | (Total) | Total |
| | N = 20 | N = 21 | N = 41 |
| eGFR (mL/min/1.73 m²) | | | |
| n | 20 | 21 | 41 |
| Mean | 47.63 | 44.73 | 46.14 |
| (SD) | (8.77) | (8.70) | (8.75) |
| Median | 48.05 | 45.10 | 46.70 |
| (Min, Max) | (30.7, 59.6) | (30.3, 58.2) | (30.3, 59.6) |
| eGFR (mL/min/1.73 m²) | | | |
| n | 20 | 21 | 41 |
| 30-44 | 6 | 10 | 16 |
| | (30.0%) | (47.6%) | (39.0%) |
| 45-59 | 14 | 11 | 25 |
| | (70.0%) | (52.4%) | 61.0% |
| HbA1c (%) | | | |
| n | 20 | 21 | 41 |
| Mean | 7.88 | 7.85 | 7.87 |
| | (1.27) | (0.74) | (1.02) |
| Median | 7.25 | 7.90 | 7.80 |
| (Min, Max) | (6.6, 10.3) | (6.5, 9.7) | (6.5, 10.3) |
| Fasting Glucose (mg/dL) | | | |
| n | 20 | 21 | 41 |
| Mean | 177.9025 | 166.5992 | 172.1130 |
| (SD) | (50.6567) | (26.8536) | (40.1519) |
| Median | 177.9475 | 164.8830 | 170.2890 |
| (Min, Max) | (107.19, 293.726) | (119.833, 233.359) | (107.219, 293.726) |
| Duration of T2 DM (yr) | | | |
| n | 20 | 21 | 41 |
| Mean | 15.905 | 16.296 | 16.105 |
| (SD) | (7.161) | (8.546) | (7.804) |
| Median | 17.000 | 15.000 | 17.000 |
| (Min, Max) | (3.00, 31.00) | (2.30, 29.00) | (2.30, 31.00) |
| Insulin Dose (U/day) | | | |
| n | 20 | 21 | 41 |
| Mean | 57.55 | 50.52 | 53.95 |
| (SD) | (49.37) | (61.96) | (55.59) |
| Median | 60.00 | 30.00 | 46.00 |
| (Min, Max) | (0.0, 167.0) | (0.0, 250.00) | (0.0, 250.0) |
| Oral Anti-glycemic Medication at Screening | | | |
| n | 12 | 16 | 28 |
| Yes | 12 | 16 | 28 |
| | (100%) | (100%) | (100%) |
| No | 0 | 0 | 0 |

Max = maximum;
Min = minimum;
SD = standard deviation
^{a}5/6 subjects in the Placebo Insulin Dose < 20 U/day group and 6/7 subjects in the MEDI0382 Insulin Dose < 20 U/day group were only on oral antidiabetic treatment.
Note:
eGFR ‘30-44’ is eGFR >30 and <45.
Note:
eGFR ‘45-59’ is eGFR >45 and <60.

The distribution of subjects was skewed towards the eGFR 45-59 mL/min/1.73 m² subgroup within the placebo group, while the Cotadutide group had approximate equal distribution of subjects in the eGFR subgroups and more subjects than placebo in the eGFR group 30-45 mL/min/1.73 m².

Comparing subgroups by insulin dose, mean baseline fasting glucose, and duration of T2DM were higher in the Insulin Dose ≥20 U/day subgroup, and more subjects in the Insulin Dose ≥20 U/day subgroup had an eGFR in the 30-45 mL/min/1.73 m² range. Differences in baseline HbA1c were also seen across the different insulin subgroups, likely due to the challenges of managing patients with renal impairment with insulin therapy.

Subjects did not have any unexpected physical examination findings or unexpected findings in medical history.

### (B) Study Design

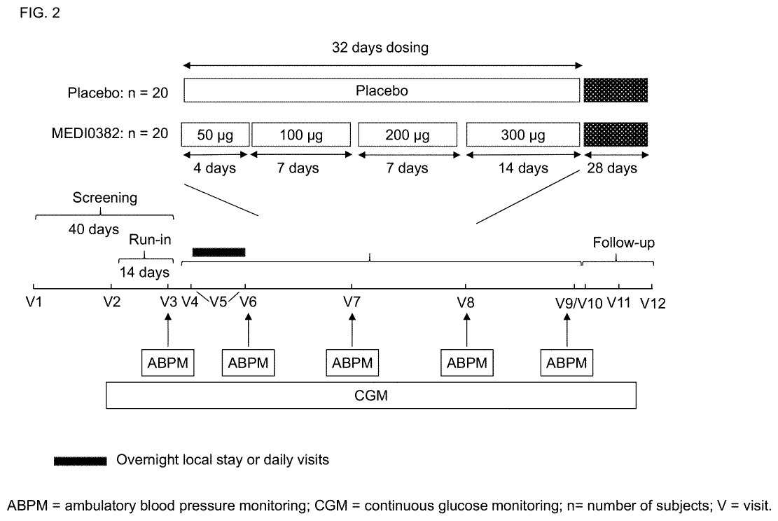

A flow diagram of the proposed study is provided in FIG. 2. In this study,

Cotadutide was titrated from 50 μg up to 300 μg and administered once daily by subcutaneous (SC) injection over 32 days. The study had a 14-day run-in period of diet and exercise and continuous glucose monitoring (CGM), a 32-day treatment period, and a 28-day follow-up period.

Subjects were randomized using a 1:1 ratio receive either Cotadutide or placebo. The randomization was stratified according to whether the subject was on insulin at a dose of at least 20 units per day or either not on insulin or at a dose of less than 20 units per day, with at least 50% of the subject randomized receiving insulin at a dose of at least 20 units per day.

Cotadutide was administered at 50 μg once daily for 4 days, followed by 100 μg daily for 7 days, 200 μg daily for 7 days, and 300 μg daily for 14 days (n=20). Placebo was administered one daily for 32 days (n=20). Cotadutide and placebo were administered in the morning. Dosing commenced following predose baseline vital signs, blood tests (including HbA1c), an electrocardiogram (ECG), body weight measurement, and bioimpedance spectroscopy (BIS). Subjects then returned for daily dosing or remained overnight locally until Day 5, and thereafter, had visits at weekly intervals until a maintenance dose of 300 μg was established. Thus, Cotadutide or placebo was administered at the study site by study personal for the first four doses and on site on Days 5, 12, 19, and 32. At all other times, subjects preformed dosing at home.

### Mixed-Meal Tolerance Test

The effect of Cotadutide titrated up to a dose level of 300 μg on glucose control vs placebo after 32 days of treatment was assessed by a standardized mixed-meal tolerance test (MMTT) to determine the percentage change in glucose from area under the concentration-time curve (AUC) from baseline (Day −5) to the end of 32 days of treatment.

Following a minimum 8-hour fast, the subject underwent an MMTT, which involved the consumption of a standardized liquid meal (Ensure Plus, a nutritional supplement containing the components of fat, carbohydrate, and protein, which make up a standard MMTT) within 15 minutes, and timed serial blood samples were obtained for measurement of glucose through 240 minutes after consumption of the standardized meal (with no additional food intake during this time). Blood was drawn within 15 minutes before consuming the standardized liquid meal (i.e., “0 minutes”), and at 15, 30, 45, 60, 90, 120, 180, and 240 minutes (±5 minutes) after consumption. Blood sampling occurred as close as possible to the specified times for the MMTT. At Day 32, the serial blood sampling begins 2.5 hours after Cotadutide or placebo administration.

Subjects who used a quick acting or pre-mixed form of insulin with breakfast received their usual/current dose of breakfast-time insulin with the Ensure Plus milkshake i.e., dose reduction of insulin was not required. Subjects who carbohydrate counted or varied insulin dose in relation to meal type were advised that the Ensure Plus milkshake contained approximately 44.4 g of carbohydrate, 10.8 g of fat, and 13.8 g of protein and to adjust their insulin dose on this basis. The timing of insulin administration in relation to the Ensure Plus milkshake consumption was in accordance with the usual regimen used by the subject, e.g., if a subject injected insulin 30 minutes before breakfast, they were to inject insulin 30 minutes before the Ensure Plus milkshake.

The investigators also considered whether a dose reduction in insulin and/or sulfonylurea or glitinide was required for this fasting assessment on Day 32.

### Blood Tests

Blood samples were collected to evaluate blood glucose (fasting) and HbA1c levels.

### Weight

Weight was measured after the subject had toileted and removed bulky clothing, including shoes. Whenever possible, the same calibrated scale was used for each measurement for any given subject.

### Continuous Glucose Monitoring

A Freestyle Libre® Pro CGM device was used to measure interstitial glucose levels. The Freestyle Libre® Pro CGM device measures interstitial glucose levels every 15 minutes for 2 weeks continuously and does not require any calibration or periodic near-touch/blue-tooth connections with the device to perform this function. The Freestyle Libre® Pro CGM device does not permit flash glucose measurements. Data generated by CGM was used at visits to adjust insulin doses and other treatments.

The CGM sensor, which is a small plastic circular device of 35 mm diameter and 5 mm depth, was applied to the back of the upper arm. Subjects wore the CGM sensor continuously up until the time of a sensor change, which was to occur within 14 days, and they were advised that they could bathe and shower, and swim in up to 3 m depth for up to 30 minutes while wearing the CGM sensor. When the CGM sensor was removed, a new CGM sensor could be reapplied, ideally close to the original site, but with the subject's site preference taken into account. CGM sensors were single-use only.

If a subject was unable to tolerate wearing the CGM sensor for the entire duration of the study, the sensor was removed; but the subject was to remain in the study with or without continued CGM and could instead be instructed to monitor capillary plasma glucose measurements four times per day (before meals and before bedtime) and record these levels in the diary provided.

### Ambulatory Blood Pressure Monitoring

After instruction, subjects set up and applied an ambulatory blood pressure monitoring (ABPM) device. The subjects were advised to undergo normal daily activities while wearing the cuff and to avoid any strenuous forms of activity, bathing, or showering while wearing the cuff. The subjects were advised to remain still during the measurement with the arm relaxed at heart level. During ABPM, systolic BP, diastolic BP, heart rate pressure, heart rate, and mean arterial pressure readings were recorded over a period of 24 hours.

### Bioimpedance Spectroscopy Measurements

Assessment of body composition, including but not limited to extracellular and intracellular volume and total body water, were conducted with bioimpedance spectroscopy using the SFB7 device produced by ImpediMed. Bioimpedance spectroscopy measures were not conducted in any subject with a pacemaker or implantable electronic device, e.g., an implantable cardiac defibrillator, as described in the manufacturer's instructions.

Bioimpedance spectroscopy was performed prior to dosing on Visit 4 (Day 1; Baseline) and following dosing on Visits 6 (Day 5), 7 (Day 12), 8 (Day 19), and 9 (Day 32). Subjects were to void their bladder and abstain from exercising 2 hours prior to the assessment. The subjects laid in a supine position 5 minutes before the measurement, while not touching any metal surfaces. The subjects' limbs were not crossed, the legs were completely separated, and the arms were not to touch the torso. For subjects who could not effectively separate their inner thighs, insulating material (e.g., a towel) may have been between the legs. The bioimpedance spectroscopy was set to make three repeat measurements. The subject was to remain still and relaxed during the measurement. The points of contact for the electrodes were marked on the subject's skin and the same positions used at all occasions. Preferably, electrodes were connected to the right side of the body (hand and foot according to the manual instructions for SFB7). The bioimpedance spectroscopy machine was charged and not connected to main power during use.

### Pharmacokinetic Evaluations

Blood was collected to evaluate to pharmacokinetics (PK) of Cotadutide in plasma. The PK was measured using a validated liquid chromatography-tandem mass spectrometry (LC/MS-MS) method.

### Immunological Evaluations and Adverse Events

Blood samples were collected to evaluate anti-drug antibody (ADA) response to Cotadutide. A screening assay in the form of a traditional ligand-binding “birding” assay using electrochemiluminescence was used to determine ADA-positive samples.

Only serious adverse events (SAEs) associated with protocol-related procedures were collected form time of signature of informed consent until the stat of the treatment period. All adverse events (AEs) were collected during the treatment period and follow-up period (Day 60±5). AEs and SAEs were graded by severity and relationship to Cotadutide or placebo, and SAEs were assessed for relationship to protocol procedures.

### (C) Results

Clinically meaningful and statistically significantly results were observed for the Cotadutide group compared with the placebo group.

### Percent Change in MMTT Plasma Glucose AUC

The change in the mixed-meal tolerance test (MMTT) plasma glucose AUC from the baseline (Day −5) to end of 32 days of treatment was evaluated in the ITT population using an analysis of covariance (ANCOVA) model. The model included fixed effect of treatment and baseline AUC as a covariate. The difference of the percent change in glucose AUC between the two treatment arms was compared with a two-sided significance level of 0.10.

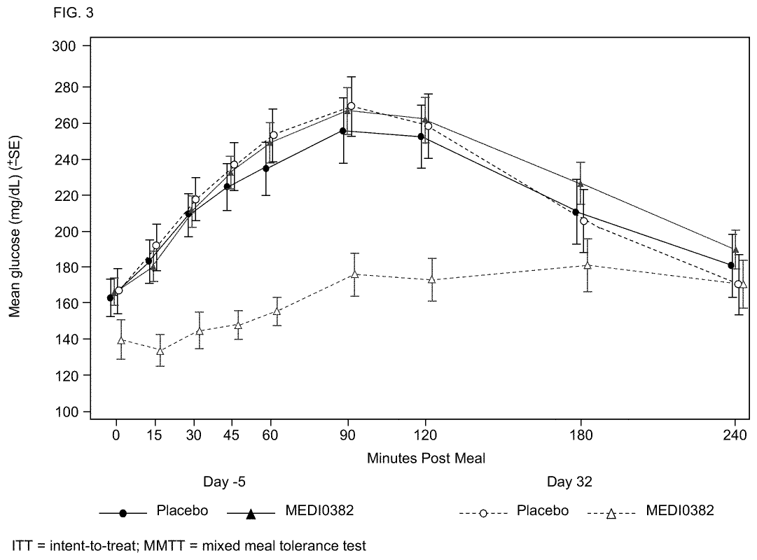

Overall postprandial plasma glucose concentrations on Day 32 were lower than placebo from pre-standardized liquid meal to 180 minutes post standardized liquid meal (FIG. 3). A statistically significant least squares (LS) mean percentage reduction from baseline to Day 32 in MMTT plasma glucose AUC_{0-4 h} was observed in the Cotadutide dose group (−26.71%) compared with the placebo group (3.68%) (p <0.001) (Table 3).

TABLE 3
Percent Change in MMTT Plasma Glucose AUC_{4 h} from Day −5 to Day 32 - ITT Population

| AUC4 h (hr*mg/dL) | | | | | |
|---|---|---|---|---|---|
| Placebo | MEDI0382 | Placebo | MEDI0382 | | |
| (Insulin | (Insulin | (Insulin | (Insulin | | |
| Dose ≥ 20 | Dose ≥ 20 | Dose < 20 | Dose < 20 | Placebo | MEDI0382 |
| U/day) | U/day) | U/day^{b}) | U/day^{b}) | (Total) | (Total) |
| N = 14 | N = 14 | N = 6 | N = 7 | N = 20 | N = 21 |

Baseline (Day −5)

| | | | | | | |
|---|---|---|---|---|---|---|
| N | 14 | 14 | 6 | 7 | 20 | 21 |
| Mean | 956.35 | 951.12 | 727.56 | 922.75 | 887.71 | 941.66 |
| (SD) | (255.84) | (191.77) | (159.62) | (200.85) | (251.12) | (190.25) |
| Median | 880.06 | 932.38 | 691.36 | 928.93 | 864.08 | 928.93 |
| (Min, Max) | (606.24, 1449.48) | (633.93, 1324.24) | (564.03, 927.53) | (650.97, 1237.48) | (564.03, 1449.48) | (633.93, 1324.24) |

Day 32

| | | | | | | |
|---|---|---|---|---|---|---|
| N | 14 | 11 | 6 | 7 | 20 | 18 |
| Mean | 946.88 | 679.23 | 840.72 | 594.85 | 915.023 | 646.42 |
| (SD) | (272.16) | (201.02) | (278.05) | (115.51) | (271.14) | (173.99) |
| Median | 848.18 | 700.08 | 765.70 | 599.41 | 817.62 | 619.33 |
| (Min, Max) | (582.95, 1421.48) | (317.18, 1040.87) | (592.18, 1377.48) | (451.33, 762.86) | (582.95, 1421.48) | (317.18, 1040.87) |

Percent Change from Baseline

| | | | | | | |
|---|---|---|---|---|---|---|
| N | 14 | 11 | 6 | 7 | 20 | 18 |
| Mean | 0.19 | −25.00 | 14.85 | −31.99 | 4.589 | −27.719 |
| (SD) | (19.67) | (23.09) | (20.21) | (23.71) | (20.48) | (22.90) |
| Median | −4.91 | −22.850 | 8.41 | −36.99 | 2.80 | −27.45 |
| (Min, Max) | (−28.80, 44.93) | (−76.05, 10.43) | (−5.56, 48.51) | (−56.15, 9.64) | (−28.80, 48.51) | (−76.05, 10.43) |
| LS Mean | 0.44 | −25.313 | 9.21 | −27.16 | 3.68 | −26.71 |
| % Changea | (−8.74, 9.62) | (−35.67, −14.96) | (−7.55, 25.98) | (−42.51, −11.81) | (−3.79, 11.15) | (−34.58, −18.83) |
| (90% CI) | | | | | | |
| Difference in | — | −25.751 | — | −36.37 | — | −30.38 |
| LS Mean | | (−39.59, −11.91) | | (−60.66, −12.08) | | (−41.27, −19.50) |
| % Change (vs | | | | | | |
| Placebo)a | | | | | | |
| (90% CI) | | | | | | |
| P-value for | — | 0.004 | — | 0.022 | — | <0.001 |
| Difference in | | | | | | |
| LS Mean | | | | | | |
| % Change (vs | | | | | | |
| Placebo)a | | | | | | |

ANCOVA = analysis of covariance;
AUC = area under the concentration-time curve;
CI = confidence interval;
ITT = intent-to-treat;
LS = least squares;
Max = maximum;
Minminimum;
MMTT = mixed meal tolerance test;
SD = standard deviation
aLS Mean, its associated CI and P-value from ANCOVA with effect for treatment group and value at the Day −5 AUC as a covariate.
^{b}5/6 subjects in the Placebo Insulin Dose < 20 U/day group and 6/7 subjects in the MEDI0382 Insulin Dose < 20 U/day group were only on oral antidiabetic treatment.

The postprandial glucose reduction was balanced upon subgroup comparisons: Insulin Dose ≥20 U/day vs Insulin Dose <20 U/day (Table 3) and eGFR 45-59 mL/min/1.73 m² vs eGFR 30-44 mL/min/1.73 m² (Table 4). Of the subjects on Insulin Dose <20 U/day, 5/6 subjects on Placebo and 6/7 subjects on Cotadutide were treated with oral antidiabetic treatment but not treated with insulin.

TABLE 4
Percent Change in MMTT Plasma Glucose AUC_{4 h} from
Day −5 to Day 32 - ITT Population, eGFR subgroup analysis

| AUC4 h (hr*mg/dL) | | | |
|---|---|---|---|
| Placebo | MEDI0382 | Placebo | MEDI0382 |
| (eGFR 30-44 | (eGFR 30-44 | (eGFR 45-59 | (eGFR 45-59 |
| mL/min/1.73 m²) | mL/min/1.73 m²) | mL/min/1.73 m²) | mL/min/1.73 m²) |
| N = 6 | N = 10 | N = 14 | N= 11 |

Baseline (Day −5)

| | | | | |
|---|---|---|---|---|
| N | 6 | 10 | 14 | 11 |
| Mean | 1001.42 | 924.59 | 838.99 | 957.19 |
| (SD) | (233.62) | (217.37) | (250.31) | (171.19) |
| Median | 961.05 | 897.15 | 786.45 | 951.30 |
| (Min, Max) | (695.7, 1375.5) | (633.9, 1324.2) | (564.0, 1449.5) | (704.6, 1262.2) |

Day 32

| | | | | |
|---|---|---|---|---|
| N | 6 | 7 | 14 | 11 |
| Mean | 1006.02 | 618.21 | 876.04 | 664.37 |
| (SD) | (343.30) | (171.03) | (238.04) | (181.66) |
| Median | 1026.25 | 700.10 | 811.20 | 605.70 |
| (Min, Max) | (582.9, 1377.5) | (317.2, 762.9) | (592.2, 1421.5) | (477.1, 1040.9) |

Percent Change from Baseline

| | | | | |
|---|---|---|---|---|
| N | 6 | 7 | 14 | 11 |
| Mean | 0.35 | −24.41 | 6.40 | −29.83 |
| (SD) | (27.70) | (31.43) | (17.49) | (16.90) |
| Median | −8.50 | −22.90 | 5.70 | −37.00 |
| (Min, Max) | (−28.8, 48.5) | (−76.0, 10.4) | (−17.9, 44.9) | (−56.2, −4.0) |
| LS Mean | 4.05 | −27.59 | 4.92 | −27.95 |
| % Change^{a} | (−15.94, 24.04) | (−46.06, −9.12) | (−2.74, 12.59) | (−36.64, −19.26) |
| (90% CI) | | | | |
| Difference | — | −31.64 | — | −32.87 |
| in LS | | (−59.23, −4.05) | | (−44.68, −21.07) |
| Mean% | | | | |
| Change (vs | | | | |
| Placebo)^{a} | | | | |
| (90% CI) | | | | |
| P-value for | — | 0.064 | — | <0.001 |
| Difference | | | | |
| in LS Mean | | | | |
| % Change | | | | |
| (vs Placebo)^{a} | | | | |

ANCOVA = analysis of covariance;
AUC = area under the concentration-time curve;
CI = confidence interval;
eGFR = estimated glomerular filtration rate;
ITT = intent-to-treat;
LS = least squares;
Max = maximum;
Min = minimum;
MMTT = mixed meal tolerance test;
SD = standard deviation
^{a}LS Mean, its associated CI and P-value from ANCOVA with effect for treatment group and value at the Day −5 AUC as a covariate.

### Change in HbA1c

A statistically significant LS mean reduction from baseline to Day 32 in HbA1c was observed in the Cotadutide dose group compared with the placebo group (−0.65% vs 0.01%, p <0.001; Table 5). LS mean reduction of HbA1c from baseline was balanced between subjects in the Insulin Dose <20 U/day subgroup compared to Insulin Dose ≥20 U/day subgroup. However, only the Insulin Dose ≥20 U/day subgroup reached statistical significance comparing Cotadutide to placebo (−0.57% vs −0.02%, p <0.001; Table 5).

TABLE 5
Change in HbA1c (%) from Day 1 to Day 32 - ITT Population

| Hemoglobin A1C (%) | | | | | |
|---|---|---|---|---|---|
| Placebo | MEDI0382 | Placebo | MEDI0382 | | |
| (Insulin | (Insulin | (Insulin | (Insulin | | |
| Dose ≥ 20 | Dose ≥ 20 | Dose < 20 | Dose < 20 | Placebo | MEDI0382 |
| U/day) | U/day) | U/day^{b}) | U/day^{b}) | (Total) | (Total) |
| N = 14 | N = 14 | N = 6 | N = 7 | N = 20 | N = 21 |

Baseline (Day 1)

| | | | | | | |
|---|---|---|---|---|---|---|
| N | 14 | 14 | 6 | 7 | 20 | 21 |
| Mean | 8.229 | 7.893 | 7.067 | 7.771 | 7.880 | 7.852 |
| (SD) | (1.356) | (0.760) | (0.446) | (0.748) | (1.268) | (0.739) |
| Median | 7.850 | 7.850 | 7.000 | 7.900 | 7.250 | 7.900 |
| (Min, Max) | (6.60, 10.30) | (6.70, 9.70) | (6.70, 7.90) | (6.50, 8.50) | (6.60, 10.30) | (6.50, 9.70) |

Day 32

| | | | | | | |
|---|---|---|---|---|---|---|
| N | 14 | 12 | 6 | 7 | 20 | 19 |
| Mean | 8.186 | 7.392 | 7.200 | 6.943 | 7.890 | 7.226 |
| (SD) | (1.249) | (0.668) | (0.701) | (0.476) | (1.188) | (0.631) |
| Median | 7.650 | 7.300 | 7.150 | 6.900 | 7.450 | 7.200 |
| (Min, Max) | (7.10, 10.50) | (6.50, 9.20) | (6.20, 8.20) | (6.30, 7.50) | (6.20, 10.50) | (6.30, 9.20) |

Change from Baseline

| | | | | | | |
|---|---|---|---|---|---|---|
| N | 14 | 12 | 6 | 7 | 20 | 19 |
| Mean | −0.043 | −−0.550 | 0.133 | −0.829 | 0.010 | −0.653 |
| (SD) | (0.372) | (0.353) | (0.659) | (0.553) | (0.464) | (0.444) |
| Median | −−0.050 | −−0.500 | 0.050 | −−0.900 | −−0.050 | −−0.600 |
| (Min, Max) | (−0.70, 0.50) | (−1.10, 0.00) | (−0.70, 1.10) | (−1.60, 0.00) | (−0.70, 1.10) | (−1.60, 0.00) |
| LS Mean | −0.02 | −0.57 | −0.06 | −0.66 | 0.01 | −0.65 |
| Change^{a} | (−0.18, 0.13) | (−0.74, −0.41) | (−0.50, 0.37) | (−1.05, −0.27) | (−0.15, 0.17) | (−0.82, −0.49) |
| (90% CI) | | | | | | |
| Difference | — | −0.55 | — | −0.59 | — | −0.66 |
| in LS | | (−0.77, −0.32) | | (−1.22, 0.03) | | (−0.89, −0.44) |
| Mean Change | | | | | | |
| (vs Placebo)^{a} | | | | | | |
| (90% CI) | | | | | | |
| P-value for | — | <0.001 | — | 0.117 | — | <0.001 |
| Difference | | | | | | |
| in LS | | | | | | |
| Mean Change | | | | | | |
| (vs Placebo)^{a} | | | | | | |

ANCOVA = analysis of covariance;
CI = confidence interval;
ITT = intent-to-treat;
LS = least squares;
Max = maximum;
Min = minimum;
SD = standard deviation
^{a}LS Mean, its associated CI and P-value from ANCOVA with effect for treatment group and baseline value as a covariate.
^{b}5/6 subjects in the Placebo Insulin Dose < 20 U/day group and 6/7 subjects in the MEDI0382 Insulin Dose < 20 U/day group were only on oral antidiabetic treatment.

### Change in Fasting Glucose

Fasting glucose was measured from baseline (Day 1) to the end of treatment (Day 32). A numerical LS mean reduction in fasting plasma glucose was observed in the Cotadutide dose group compared with the placebo group (−19.55 mg/dL vs 0.60 mg/dL, p=0.089; Table 6). LS mean reduction of fasting glucose from baseline was more apparent in subjects in the Insulin Dose <20 U/day subgroup compared to Insulin Dose ≥20 U/day subgroup. Furthermore, comparison of LS mean reduction of fasting glucose in the Insulin Dose <20 U/day subgroup comparing Cotadutide to placebo reached statistical significance (−32.24 mg/dL vs −4.13 mg/dL, p=0.043); Table 6).

TABLE 6
Change in Fasting Glucose (mg/dL) from Day 1 to Day 32 - ITT Population

| Glucose (mg/dL) | | | | | |
|---|---|---|---|---|---|
| Placebo | MEDI0382 | Placebo | MEDI0382 | | |
| (Insulin | (Insulin | (Insulin | (Insulin | | |
| Dose ≥ 20 | Dose ≥ 20 | Dose < 20 | Dose < 20 | Placebo | MEDI0382 |
| U/day) | U/day) | U/day^{b}) | U/day^{b}) | (Total) | (Total) |
| N = 14 | N = 14 | N = 6 | N = 7 | N = 20 | N = 21 |

Baseline (Day 1)

| | | | | | | |
|---|---|---|---|---|---|---|
| N | 14 | 14 | 6 | 7 | 20 | 21 |
| Mean | 185.28 | 171.13 | 160.68 | 157.55 | 177.90 | 166.60 |
| (SD) | (54.76) | (30.55) | (38.01) | (15.44) | (50.66) | (26.85) |
| Median | 179.30 | 173.44 | 159.48 | 154.07 | 177.95 | 164.88 |
| (Min, Max) | (107.2, 293.7) | (119.8, 233.4) | (115.3, 202.7) | (141.5, 181.1) | (107.2, 293.7) | (119.8, 233.4) |

Day 32

| | | | | | | |
|---|---|---|---|---|---|---|
| N | 14 | 12 | 6 | 7 | 20 | 19 |
| Mean | 185.54 | 159.03 | 156.02 | 125.75 | 176.69 | 146.77 |
| (SD) | (45.54) | (55.14) | (34.65) | (21.84) | (43.90) | (47.85) |
| Median | 178.40 | 141.91 | 150.92 | 125.24 | 168.04 | 139.66 |
| (Min, Max) | (125.2, 273.9) | (88.3, 275.7) | (113.5, 215.3) | (103.6, 160.4) | (113.5, 273.9) | (88.3, 275.7) |

Change from Baseline

| | | | | | | |
|---|---|---|---|---|---|---|
| N | 14 | 12 | 6 | 7 | 20 | 19 |
| Mean | 0.26 | −9.39 | −4.66 | −31.79 | −1.22 | −17.64 |
| (SD) | (36.34) | (47.64) | (24.93) | (20.25) | (32.75) | (40.58) |
| Median | 4.05 | −9.01 | 2.25 | −39.64 | 2.25 | −19.82 |
| (Min, Max) | (−77.5, 40.5) | (−112.6, 55.9) | (−36.9, 19.8) | (−56.8, 2.7) | (−77.5, 40.5) | (−112.6, 55.9) |
| LS Mean | 2.74 | −12.28 | −4.13 | −32.24 | 0.60 | −19.55 |
| Change^{a} | (−15.79, 21.27) | (−32.32, 7.77) | (−20.26, 11.99) | (−47.16, −17.31) | (−12.89, 14.08) | (−33.39, −5.71) |
| (90% CI) | | | | | | |
| Difference in | — | −15.02 | — | −28.11 | — | −20.15 |
| LS Mean | | (−42.56, 12.53) | | (−50.10, −6.12) | | (−39.61, −0.69) |
| Change (vs | | | | | | |
| Placebo)^{a} | | | | | | |
| (90% CI) | | | | | | |
| P-value for | — | 0.360 | — | 0.043 | — | 0.089 |
| Difference in | | | | | | |
| LS Mean | | | | | | |
| Change (vs | | | | | | |
| Placebo)^{a} | | | | | | |

ANCOVA = analysis of covariance;
CI = confidence interval;
ITT = intent-to-treat;
LS = least squares;
Max = maximum;
Min = minimum;
SD = standard deviation
^{a}LS Mean, its associated CI and P-value from ANCOVA with effect for treatment group and baseline value as a covariate.
^{b}5/6 subjects in the Placebo Insulin Dose < 20 U/day group and 6/7 subjects in the MEDI0382 Insulin Dose < 20 U/day group were only on oral antidiabetic treatment.

### Time Spent in Target Glucose Range

Subjects on Cotadutide had a significantly greater LS mean increase from baseline (Days −8 to −2) to the final week of treatment (Days 26 to 32) in percentage of time within a target glucose range (70 mg/dL [3.9 mmol/L] to 180 mg/dL [10 mmol/L]) over a 7-day period compared to subjects on Placebo (14.79% vs −21.23%, p=0.001; Table 7). LS mean increase in time within target glucose range was even more pronounced in subjects in the Insulin Dose <20 U/day subgroup (56.7% change vs Placebo) compared to Insulin Dose ≥20 U/day subgroup (19.6% change vs Placebo).

TABLE 7
Change in Percentage of Time CGM Glucose Within Target Range^{a} During 7-day Periods
from Baseline to End of Dosing at 100, 200, and 300 μg - ITT Population

| Percentage of Time CGM Glucose Within Target Range (%) | | | | | |
|---|---|---|---|---|---|
| Placebo | MEDI0382 | Placebo | MEDI0382 | | |
| (Insulin | (Insulin | (Insulin | (Insulin | | |
| Dose ≥ 20 | Dose ≥ 20 | Dose <20 | Dose <20 | Placebo | MEDI0382 |
| U/day) | U/day)) | U/day^{c} | U/day^{c}) | (Total) | (Total) |
| N = 14 | N = 14 | N = 6 | N = 7 | N = 20 | N = 21 |

Baseline (Days −8 to −2)

| | | | | | | |
|---|---|---|---|---|---|---|
| N | 13 | 14 | 6 | 6 | 19 | 20 |
| Mean | 48.27 | 57.45 | 77.76 | 62.70 | 57.58 | 59.02 |
| (SD) | (28.79) | (23.17) | (13.66) | (21.05) | (28.34) | (22.13) |
| Median | 55.65 | 60.49 | 77.57 | 60.27 | 62.78 | 60.49 |
| (Min, Max) | (0.0, 94.2) | (0.0, 85.3) | (62.5, 93.9) | (41.7, 93.6) | (0.0, 94.2) | (0.0, 93.6) |

100 μg Dose (Days 5 to 11)

| | | | | | | |
|---|---|---|---|---|---|---|
| N | 14 | 14 | 5 | 7 | 19 | 21 |
| Mean | 47.64 | 66.51 | 50.03 | 94.44 | 48.27 | 75.82 |
| (SD) | (25.23) | (31.44) | (34.42) | (7.09) | (26.91) | (28.97) |
| Median | 54.02 | 77.04 | 57.44 | 97.79 | 56.55 | 84.38 |
| (Min, Max) | (1.5, 81.0) | (0.0, 97.3) | (0.0, 81.5) | (80.6, 99.3) | (0.0, 81.5) | (0.0, 99.3) |

Change from Baseline

| | | | | | | |
|---|---|---|---|---|---|---|
| N | 12 | 13 | 5 | 6 | 17 | 19 |
| Mean | −3.59 | 2.53 | −24.51 | 31.19 | −9.74 | 11.58 |
| (SD) | (14.10) | (27.42) | (35.56) | (21.60) | (23.44) | (28.61) |
| Median | −3.29 | 12.04 | −18.60 | 33.99 | −4.47 | 14.94 |
| (Min, Max) | (−37.6, 16.2) | (−59.1, 27.8) | (−79.1, 18.8) | (5.7, 57.3) | (−79.1, 18.8) | (−59.1, 57.3) |
| LS Mean | −4.73 | 3.59 | −18.58 | 26.25 | −10.49 | 12.25 |
| Change^{b} | (−15.78, 6.31) | (−7.01, 14.19) | (−40.14, 2.98) | (6.68, 45.82) | (−20.77, −0.20) | (2.52, 21.98) |
| (90% CI) | | | | | | |
| Difference in | — | 8.32 | — | 44.84 | — | 22.74 |
| LS Mean | | (−7.19, 23.83) | | (14.82, 74.85) | | (8.55, 36.92) |
| Change (vs | | | | | | |
| Placebo)^{b} | | | | | | |
| (90% CI) | | | | | | |
| P-value for | — | 0.367 | — | 0.024 | — | 0.011 |
| Difference in | | | | | | |
| LS Mean | | | | | | |
| Change (vs | | | | | | |
| Placebo)^{b} | | | | | | |

200 μg Dose (Days 12 to 18)

| | | | | | | |
|---|---|---|---|---|---|---|
| N | 14 | 14 | 6 | 7 | 20 | 21 |
| Mean | 48.40 | 73.53 | 70.36 | 89.01 | 54.99 | 78.69 |
| (SD) | (26.85) | (21.25) | (25.56) | (12.47) | (27.78) | (19.90) |
| Median | 51.40 | 79.59 | 76.68 | 96.38 | 57.89 | 80.69 |
| (Min, Max) | (8.9, 88.5) | (21.8, 93.7) | (20.8, 90.7) | (69.1, 100.0) | (8.9, 90.7) | (21.8, 100.0) |

Change from Baseline

| | | | | | | |
|---|---|---|---|---|---|---|
| N | 12 | 13 | 6 | 6 | 18 | 19 |
| Mean | −3.94 | 11.20 | −7.41 | 24.48 | −5.10 | 15.39 |
| (SD) | (22.70) | (17.40) | (21.45) | (17.23) | (21.72) | (18.01) |
| Median | −4.74 | 11.27 | −2.87 | 23.79 | −3.34 | 14.44 |
| (Min, Max) | (−39.5, 42.5) | (−28.9, 40.9) | (−41.7, 17.1) | (4.9, 47.0) | (−41.7, 42.5) | (−28.9, 47.0) |
| LS Mean | −5.80 | 12.92 | −3.97 | 21.04 | −5.34 | 15.62 |
| Change^{b} | (−15.25, 3.65) | (3.85, 21.99) | (−18.65, 10.72) | (6.36, 35.73) | (−12.77, 2.10) | (8.39, 22.86) |
| (90% CI) | | | | | | |
| Difference in | — | 18.72 | — | 25.01 | — | 20.96 |
| LS Mean | | (5.46, 31.99) | | (3.25, 46.77) | | (10.58, 31.34) |
| Change (vs | | | | | | |
| Placebo)^{b} | | | | | | |
| (90% CI) | | | | | | |
| P-value for | — | 0.024 | — | 0.064 | — | 0.002 |
| Difference in | | | | | | |
| LS Mean | | | | | | |
| Change (vs | | | | | | |
| Placebo)^{b} | | | | | | |

300 μg Dose (Days 26 to 32)

| | | | | | | |
|---|---|---|---|---|---|---|
| N | 14 | 12 | 6 | 7 | 20 | 19 |
| Mean | 45.36 | 71.69 | 29.69 | 89.31 | 40.66 | 78.18 |
| (SD) | (29.87) | (28.79) | (41.08) | (11.18) | (33.30) | (24.99) |
| Median | 48.84 | 83.04 | 8.30 | 95.35 | 43.24 | 83.51 |
| (Min, Max) | (0.0, 81.9) | (0.0, 97.4) | (0.0, 95.0) | (71.0, 97.2) | (0.0, 95.0) | (0.0, 97.4) |

Change from Baseline

| | | | | | | |
|---|---|---|---|---|---|---|
| N | 12 | 11 | 6 | 6 | 18 | 17 |
| Mean | −7.38 | 8.44 | −48.08 | 25.61 | −20.95 | 14.50 |
| (SD) | (26.11) | (30.29) | (46.47) | (21.38) | (38.28) | (28.07) |
| Median | 0.75 | 13.31 | −60.98 | 23.18 | −14.84 | 18.31 |
| (Min, Max) | (−52.2, 26.4) | (−77.7, 34.8) | (−93.9, 32.2) | (3.0, 55.5) | (−93.9, 32.2) | (−77.7, 55.5) |
| LS Mean | −9.19 | 10.40 | −−39.60 | 17.13 | −21.23 | 14.79 |
| Change^{b} | (−22.82, 4.44) | (−3.85, 24.66) | (−64.65, −14.55) | (−7.92, 42.18) | (−33.13, −9.32) | (2.54, 27.04) |
| (90% CI) | | | | | | |
| Difference in | — | 19.59 | — | 56.73 | — | 36.02 |
| LS Mean | | (−0.33, 39.52) | | (19.62, 93.84) | | (18.94, 53.10) |
| Change (vs | | | | | | |
| Placebo)^{b} | | | | | | |
| (90% CI) | | | | | | |
| P-value for | — | 0.105 | — | 0.021 | — | 0.001 |
| Difference in | | | | | | |
| LS Mean | | | | | | |
| Change (vs | | | | | | |
| Placebo)^{b} | | | | | | |

ANCOVA = analysis of covariance;
COM = continuous glucose monitoring;
CI = confidence interval;
ITT = intent-to-treat;
LS = least squares;
Max = maximum;
Min = minimum;
SD = standard deviation
^{a} Target glucose range = 70 mg/dL [3.9 mmol/L] to 180 mg/dL [10 mmol/L]
^{b}LS Mean, its associated CI and P-value from ANCOVA with effect for treatment group and baseline value as a co variate.
^{c}5/6 subjects in the Placebo Insulin Dose < 20 U/day group and 6/7 subjects in the MEDI0382 Insulin Dose < 20 U/day group were only on oral antidiabetic treatment.

### Change in Body Weight

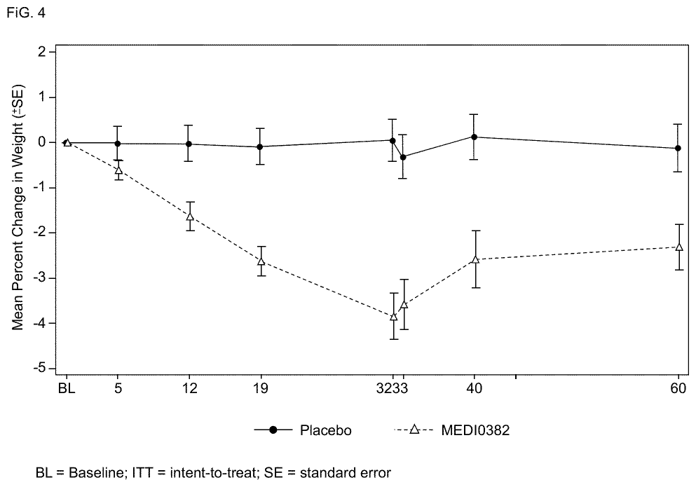

A statistically significant LS mean reduction from Day 1 to Day 33 in absolute and percentage body weight was observed in the Cotadutide dose group compared with the Placebo group (−3.41 kg [−3.69%] vs −0.13 kg [−0.21%], p <0.001; Table 8). Differences in weight reduction in the cotadutide dose group vs placebo can be seen throughout the study period starting from Day 5 (FIG. 4). Greater weight loss was observed in the <20 U/day Insulin group (−5.38%) vs the ≥20 U/day Insulin group (−2.69%).

TABLE 8
Percent Change and Absolute Change in Weight (kg) Over Time - ITT Population

| Weight | | | | | |
|---|---|---|---|---|---|
| Placebo | MEDI0382 | Placebo | MEDI0382 | | |
| (Insulin | (Insulin | (Insulin | (Insulin | | |
| Dose ≥ 20 | Dose ≥ 20 | Dose < 20 | Dose < 20 | Placebo | MEDI0382 |
| U/day) | U/day) | U/day^{b}) | U/day^{b}) | (Total) | (Total) |
| N = 14 | N = 14 | N = 6 | N = 7 | N = 20 | N = 21 |

Day 1 (kg)

| | | | | | | |
|---|---|---|---|---|---|---|
| N | 14 | 14 | 6 | 7 | 20 | 21 |
| Mean | 92.41 | 93.89 | 88.92 | 96.01 | 91.37 | 94.60 |
| (SD) | (17.61) | (10.26) | (11.77) | (29.04) | (15.85) | (17.96) |
| Median | 89.10 | 97.35 | 84.65 | 93.20 | 88.10 | 97.30 |
| (Min, Max) | (56.7, 120.1) | (71.8, 107.5) | (78.4, 108.8) | (69.5, 155.0) | (56.7, 120.1) | (69.5, 155.0) |

End of Dosing (Day 33) (kg)

| | | | | | | |
|---|---|---|---|---|---|---|
| N | 14 | 12 | 6 | 7 | 20 | 19 |
| Mean | 92.50 | 94.13 | 88.22 | 91.27 | 91.22 | 93.08 |
| (SD) | (18.63) | (6.71) | (11.68) | (28.87) | (16.66) | (17.53) |
| Median | 89.05 | 96.15 | 84.05 | 88.70 | 87.05 | 94.10 |
| (Min, Max) | (54.6, 121.7) | (81.7, 103.2) | (78.0, 107.8) | (63.5, 149.3) | (54.6, 121.7) | (63.5, 149.3) |

Percent Change from Day 1 (%)

| | | | | | | |
|---|---|---|---|---|---|---|
| N | 14 | 12 | 6 | 7 | 20 | 19 |
| Mean | −0.11 | −2.61 | −0.78 | −5.26 | −0.31 | −3.59 |
| (SD) | (2.39) | (2.23) | (1.37) | (1.81) | (2.12) | (2.42) |
| Median | 0.10 | −2.00 | −0.67 | −4.53 | −0.35 | −3.68 |
| (Min, Max) | (−3.7, 4.9) | (−8.1, −0.3) | (−2.5, 0.8) | (−8.6, −3.7) | (−3.7, 4.9) | (−8.6, −0.3) |
| LS Mean | −0.04 | −2.69 | −0.64 | −5.38 | −0.21 | −3.69 |
| Change^{a} | (−1.10, 1.03) | (−3.84, −1.54) | (−1.72, 0.45) | (−6.39, −4.38) | (−1.05, 0.62) | (−4.55, −2.83) |
| (90% CI) | | | | | | |
| Difference in | — | −2.66 | — | −4.74 | — | −3.47 |
| LS Mean | | (−4.23, −1.08) | | (−6.23, −3.26) | | (−4.68, −2.26) |
| Change (vs | | | | | | |
| Placebo)^{a} | | | | | | |
| P-value for | — | 0.008 | — | <0.001 | — | <0.001 |
| Difference in | | | | | | |
| LS Mean | | | | | | |
| Change (vs | | | | | | |
| Placebo)^{a} | | | | | | |

Absolute Change From Day 1 (kg)

| | | | | | | |
|---|---|---|---|---|---|---|
| N | 14 | 12 | 6 | 7 | 20 | 19 |
| Mean | 0.09 | −2.60 | −0.70 | −4.74 | −0.15 | −3.39 |
| (SD) | (2.06) | (2.31) | (1.14) | (0.88) | (1.84) | (2.16) |
| Median | 0.05 | −1.90 | −0.70 | −4.50 | −0.35 | −3.60 |
| (Min, Max) | (−3.3, 4.1) | (−8.3, −0.3) | (−2.0, 0.7) | (−6.0, −3.6) | (−3.3, 4.1) | (−8.3, −0.3) |
| LS Mean Change^{a} | 0.13 | −2.66 | −0.73 | −4.72 | −0.13 | −3.41 |
| (90% CI) | (−0.88, 1.15) | (−3.75, −1.56) | (−1.50, 0.05) | (−5.44, −4.00) | (−0.90, 0.64) | (−4.20, −2.62) |
| Difference in | — | −2.79 | — | −3.99 | — | −3.28 |
| LS Mean | | (−4.29, −1.29) | | (−5.05, −2.93) | | (−4.39, −2.18) |
| Change (vs | | | | | | |
| Placebo)^{a} | | | | | | |
| (90% CI) | | | | | | |
| P-value for | — | 0.004 | — | <0.001 | — | <0.001 |
| Difference in | | | | | | |
| LS Mean | | | | | | |
| Change (vs | | | | | | |
| Placebo)^{a} | | | | | | |

ANCOVA = analysis of covariance;
CI = confidence interval;
ITT = intent-to-treat;
LS = least squares;
Max = maximum;
Min = minimum;
SD = standard deviation
^{a}LS Mean, its associated CI and P-value from ANCOVA with effect for treatment group and value at the Day 1 as a co variate.
^{b}5/6 subjects in the Placebo Insulin Dose < 20 U/day group and 6/7 subjects in the MEDI0382 Insulin Dose < 20 U/day group were only on oral antidiabetic treatment.

### Effect of Cotadutide on Glucose Lowering

Subjects underwent continuous glucose monitoring (CGM) starting from Day −14 to Day 40 (±5 days), with sensor changes within 14-day intervals. CGM readings were used to analyze change in average CGM glucose and glucose AUC_{7 d}, coefficient of variation as a marker of glycemic variability, and time spent in target glucose range and hypoglycemia, to enable comparison across different dose levels.

Subjects on Cotadutide showed a progressively increasing difference in LS mean change from baseline to 7-day dosing periods in the percentage of time spent within target glucose range (70-180 mg/dL) as compared to subjects on placebo (100 μg: 22.74%; p=0.011; 200 μg: 20.96%, p=0.002; 300 μg [Days 19 to 25]: 35.23%, p <0.001; 300 μg [Days 26 to 32]: 36.02%, p=0.001). Over the course of the entire dosing period, subjects on Cotadutide spent a significantly greater mean percentage of time within the target glucose range of 70-180 mg/dL compared to subjects on Placebo (78.67% vs 44.89%, p <0.001; Table 9).

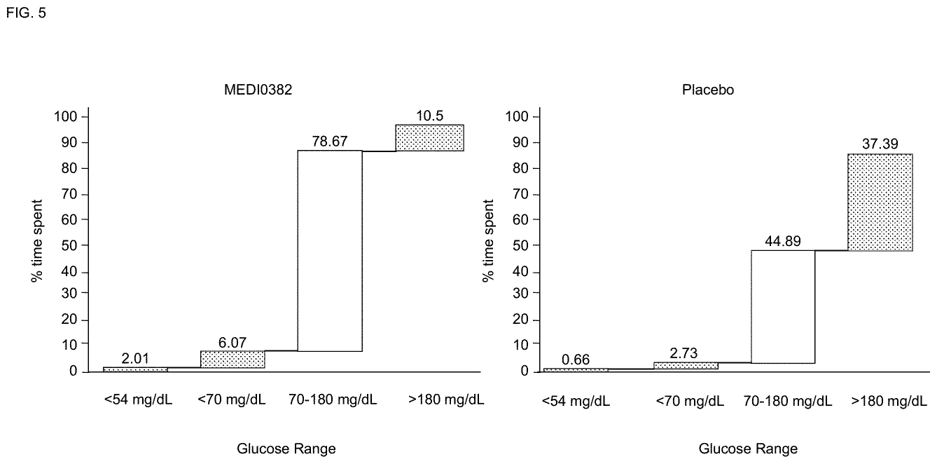

Over the course of the entire dosing period, subjects on Cotadutide spent significantly reduced mean percentage of time within hyperglycemic range of >180 mg/dL (10.50% vs 37.39%, p=0.001) as compared to placebo, especially in the Insulin Dose <20 U/day subgroup (Table 9 and FIG. 5). The decreased time in hyperglycemic range was observed from baseline to the end of each 7-day dosing period (Table 10). Less time spent in hyperglycemia was observed despite an overall reduction in insulin dose requirement in the Cotadutide arm (refer to discussion of “Effect of Cotadutide on Insulin Dose Adjustment” below and Table 15 below).

Additionally, subjects in the Cotadutide arm spent more time in hypoglycemia (glucose range <70 mg/dL) vs placebo (6.07% vs 2.73%, p=0.061) over the entire dosing period; this difference was statistically significant in those on ≥20 U/day insulin (5.68% vs 2.10%, p=0.008; Table 9). However, no statistically significant differences in time spent in hypoglycemia were observed between Cotadutide and placebo groups comparing 7-day periods from baseline to the end of each dose level (Table 11).

A statistically significant LS mean increase from baseline to the end of the 300 μg dosing period in the percentage of time spent in clinically significant hypoglycemic range (<54 mg/dL) was observed across the entire 32 day dosing period in the Cotadutide dose group compared with placebo (2.01% vs 0.66%; p=0.010) (Table 9).

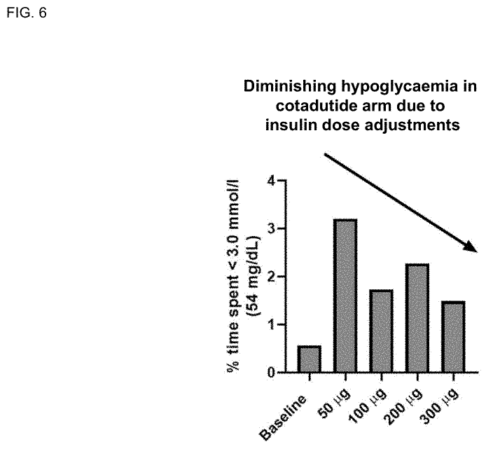

Evaluation at each dose level revealed a statistically significant greater percentage of time spent below 54 mg/dL vs Placebo at the 50 μg dose level (Table 12). However, as the dose levels were increased, less percentage of time was spent below 54 mg/dL in the Cotadutide group and the difference vs placebo was reduced; in the last week of dosing at 300 μg, there was no clinically significant difference vs placebo. The reduction in percentage time spent in clinically significant hypoglycemia was likely due to ongoing reductions in insulin and sulfonylurea doses throughout the Cotadutide dosing period (FIG. 6).

Average glucose levels showed significant reductions from baseline to the end of each 7-day dosing period in the Cotadutide dose group compared to placebo (p ≤0.003, based on LS mean change) (Table 13). The mean glucose value at the 50 μg dose was 133.7 mg/dL, at the 100 μg dose was 127.6 mg/dL, at the 200 μg dose was 131.7 mg/dL, and at the 300 μg dose (Days 26 to 32) was 125.4 mg/dL. Total glucose over each dosing period (AUC_{7 d}) was significantly decreased from baseline (Days −8 to −2) in the Cotadutide group as compared to placebo (based on LS mean change).

There was no statistically significant difference in the LS mean change in coefficient of variation of glucose from baseline to the end of each 7-day dosing period between the Cotadutide group and the placebo group. Furthermore, LS mean changes in coefficient of variation of glucose from baseline to the end of the final dosing period (Day 26-32) in subjects on Cotadutide were comparable between subjects in the Insulin Dose <20 U/day subgroup compared to Insulin Dose ≥20 U/day subgroup.

TABLE 9
Percentage of Time Spent With Various CGM Glucose
Ranges During Treatment - ITT Population

| | Placebo | MEDI0382 | Placebo | MEDI0382 | | |
|---|---|---|---|---|---|---|
| | (Insulin | (Insulin | (Insulin | (Insulin | | |
| | Dose ≥ 20 | Dose ≥ 20 | Dose < 20 | Dose < 20 | Placebo | MEDI0382 |
| | U/day) | U/day) | U/day^{a}) | U/day^{a}) | (Total) | (Total) |
| Glucose Range: | N = 14 | N =14 | N = 6 | N = 7 | N = 20 | N = 21 |
| <54 mg/dL | 0.68 | 1.93* | 0.61 | 2.15 | 0.66 | 2.01* |
| <70 mg/dL | 2.10 | 5.68* | 4.19 | 6.85 | 2.73 | 6.07 |
| 70-180 mg/dL | 46.67 | 73.09* | 40.73 | 89.85* | 44.89 | 78.67* |
| >180 mg/dL | 44.09 | 14.09* | 21.75 | 3.31 | 37.39 | 10.50* |

^{a}5/6 subjects in the Placebo Insulin Dose < 20 U/day group and 6/7 subjects in the MEDI0382 Insulin Dose < 20 U/day group were only on oral antidiabetic treatment.
*significance at p < 0.05

TABLE 10
Change in Percentage of Time CGM Glucose Within Hyperglycemic Range (>180 mg/dL [10 mmol/L]
During 7-day Periods from Baseline to End of Dosing at 100, 200, and 300 μg - ITT Population

| Percentage of time CGM Glucose in Hyperglycemia | | | | |
|---|---|---|---|---|
| 100 μg (Days 5 to 11) | | 200 μg (Days 12 to 18) | | 300 μg (Days 19 to 25) |
| Placebo | MEDI0382 | Placebo | MEDI0382 | Placebo |
| N = 20 | N = 21 | N = 20 | N = 21 | N = 20 |

Baseline (Days −8 to −2)

| | | | | | |
|---|---|---|---|---|---|
| n | 19 | 20 | 19 | 20 | 19 |
| Mean | 33.10 | 33.91 | 33.10 | 33.91 | 33.10 |
| (SD) | (27.98) | (19.92) | (27.98) | (19.92) | (27.98) |
| Median | 33.93 | 35.19 | 33.93 | 35.19 | 33.93 |
| (Min, Max) | (0.0, 91.1) | (0.0, 77.5) | (0.0, 91.1) | (0.0, 77.5) | (0.0, 91.1) |

End of Dosing Period

| | | | | | |
|---|---|---|---|---|---|
| n | 19 | 21 | 20 | 21 | 20 |
| Mean | 43.45 | 10.46 | 41.92 | 14.77 | 36.54 |
| (SD) | (28.53) | (14.10) | (29.73) | (20.69) | (31.12) |
| Median | 42.56 | 3.72 | 39.78 | 6.61 | 32.90 |
| (Min, Max) | (0.0, 98.5) | (0.0, 49.7) | (3.9, 91.1) | (0.0, 73.0) | (0.0, 91.1) |

Change from Baseline to End of Dosing Period

| | | | | | |
|---|---|---|---|---|---|
| n | 17 | 19 | 18 | 19 | 18 |
| Mean | 4.91 | −24.45 | 6.36 | −19.36 | 0.02 |
| (SD) | (16.60) | (15.59) | (21.76) | (19.66) | (27.21) |
| Median | 2.49 | −19.57 | 5.58 | −17.72 | 1.40 |
| (Min, Max) | (−18.8, 37.6) | (−57.4, −2.2) | (−45.3, 43.8) | (−54.4, 25.5) | (−63.2, 59.8) |
| LS Mean | 5.10 | −24.62 | 6.23 | −19.24 | −0.01 |
| Changea | (−0.76, 10.97) | (−30.17, −19.08) | (−1.48, 13.94) | (−26.74, −11.73) | (−7.98, 7.97) |
| (90% CI) | | | | | |
| Difference in | — | −29.72 | — | −25.47 | — |
| LS Mean | | (−37.79, −21.65) | | (−36.23, −14.71) | |
| Change (vs | | | | | |
| Placebo)a | | | | | |
| (90% CI) | | | | | |
| P-value for | — | <0.001 | — | <0.001 | — |
| Difference in | | | | | |
| LS Mean | | | | | |
| Change (vs | | | | | |
| Placebo)a | | | | | |

| | Percentage of time CGM Glucose in Hyperglycemia | | |
|---|---|---|---|
| | 300 μg (Days 19 to 25) | 300 μg (Days 26 to 32) | |
| | MEDI0382 | Placebo | MEDI0382 |
| | N = 21 | N = 20 | N = 21 |
| Baseline (Days −8 to −2) | | | |
| n | 20 | 19 | 20 |
| Mean | 33.91 | 33.10 | 33.91 |
| (SD) | (19.92) | (27.98) | (19.92) |
| Median | 35.19 | 33.93 | 35.19 |
| (Min, Max) | (0.0, 77.5) | (0.0, 91.1) | (0.0, 77.5) |
| End of Dosing Period | | | |
| n | 20 | 20 | 19 |
| Mean | 10.65 | 31.29 | 10.00 |
| (SD) | (13.95) | (31.68) | (16.81) |
| Median | 5.81 | 21.55 | 4.38 |
| (Min, Max) | (0.3, 61.7) | (0.0, 88.8) | (0.0, 71.0) |
| Change from Baseline to End of Dosing Period | | | |
| n | 18 | 18 | 17 |
| Mean | −23.50 | −5.89 | −25.49 |
| (SD) | (17.33) | (29.99) | (18.29) |
| Median | −19.64 | −6.30 | −23.12 |
| (Min, Max) | (−54.8, 1.4) | (−63.2, 51.6) | (−55.5, −2.6) |
| LS Mean | −23.47 | −6.21 | −25.15 |
| Changea | (−31.45, −15.50) | (−15.19, 2.77) | (−34.39, −15.91) |
| (90% CI) | | | |
| Difference in | −23.47 | — | −18.94 |
| LS Mean | (−34.74, −12.19) | | (−31.83, −6.05) |
| Change (vs | | | |
| Placebo)a | | | |
| (90% CI) | | | |
| P-value for | 0.001 | — | 0.018 |
| Difference in | | | |
| LS Mean | | | |
| Change (vs | | | |
| Placebo)a | | | |

ANCOVA = analysis of covariance;
CGM = continuous glucose monitoring;
CI = confidence interval;
ITT = intent-to-treat;
LS = least squares;
Max = maximum;
Min = minimum;
SD = standard deviation
aLS Mean, its associated CI and P-value from ANCOVA with effect for treatment group and baseline value as a covariate.

TABLE 11
Change in Percentage of Time CGM Glucose Within Hypoglycemic Range (<70 mg/dl [3.9 mmol/L])
During 7-day Periods from Baseline to End of Dosing at 100, 200, and 300 μg - ITT Population

| Percentage of time CGM Glucose in Hypoglycemia | | | | | | | |
|---|---|---|---|---|---|---|---|
| 100 μg (Days 5 to 11) | | 200 μg (Days 12 to 18) | | 300 μg (Days 19 to 25) | | 300 μg (Days 26 to 32) | |
| Placebo | MEDI0382 | Placebo | MEDI0382 | Placebo | MEDI0382 | Placebo | MEDI0382 |
| N = 20 | N = 21 | N = 20 | N = 21 | N = 20 | N = 21 | N = 20 | N = 21 |

Baseline (Days −8 to −2)

| | | | | | | | | |
|---|---|---|---|---|---|---|---|---|
| n | 19 | 20 | 19 | 20 | 19 | 20 | 19 | 20 |
| Mean | 4.05 | 2.07 | 4.05 | 2.07 | 4.05 | 2.07 | 4.05 | 2.07 |
| (SD) | (5.69) | (3.67) | (5.69) | (3.67) | (5.69) | (3.67) | (5.69) | (3.67) |
| Median | 1.64 | 0.45 | 1.64 | 0.45 | 1.64 | 0.45 | 1.64 | 0.45 |
| (Min, Max) | (0.0, 20.8) | (0.0, 15.5) | (0.0, 20.8) | (0.0, 15.5) | (0.0, 20.8) | (0.0, 15.5) | (0.0, 20.8) | (0.0, 15.5) |

End of Dosing Period

| | | | | | | | | |
|---|---|---|---|---|---|---|---|---|
| n | 19 | 21 | 20 | 21 | 20 | 20 | 20 | 19 |
| Mean | 3.02 | 4.20 | 3.09 | 6.53 | 2.97 | 6.67 | 3.05 | 6.55 |
| (SD) | (5.55) | (5.72) | (5.09) | (6.66) | (7.83) | (8.63) | (7.88) | (7.90) |
| Median | 0.00 | 0.89 | 0.69 | 4.17 | 0.00 | 5.28 | 0.00 | 4.23 |
| (Min, Max) | (0.0, 19.3) | (0.0, 17.0) | (0.0, 21.0) | (0.0, 20.4) | (0.0, 34.6) | (0.0, 37.6) | (0.0, 33.5) | (0.0, 26.1) |

Change from Baseline to End of Dosing Period

| | | | | | | | | |
|---|---|---|---|---|---|---|---|---|
| n | 10 | 10 | 11 | 10 | 11 | 9 | 11 | 8 |
| Mean | −1.79 | 3.51 | −2.46 | 1.47 | −2.25 | 5.10 | −2.96 | 4.66 |
| (SD) | (8.68) | (6.35) | (8.09) | (8.22) | (11.55) | (14.19) | (11.63) | (10.16) |
| Median | −3.72 | 3.39 | −2.08 | 2.22 | −4.01 | 2.60 | −3.87 | 1.07 |
| (Min, Max) | (−12.0, 14.9) | (−5.5, 14.4) | (−11.9, 16.5) | (−15.2, 16.3) | (−13.0, 30.2) | (−14.7, 34.9) | (−15.5, 29.0) | (−8.7, 22.1) |
| LS Mean | −0.54 | 2.26 | −0.99 | −0.14 | −0.85 | 3.39 | −1.81 | 3.09 |
| Changea | (−4.30, 3.22) | (−1.50, 6.02) | (−4.21, 2.22) | (−3.51, 3.24) | (−6.90, 5.20) | (−3.32, 10.10) | (−6.81, 3.19) | (−2.79, 8.98) |
| (90% CI) | | | | | | | | |
| Difference in | — | 2.80 | — | 0.86 | — | 4.25 | — | 4.91 |
| LS Mean | | (−2.66, 8.25) | | (−3.89, 5.61) | | (−4.93, 13.42) | | (−2.91, 12.72) |
| Change (vs | | | | | | | | |
| Placebo)a | | | | | | | | |
| (90% CI) | | | | | | | | |
| P-value for | — | 0.385 | — | 0.758 | — | 0.432 | — | 0.289 |
| Difference in | | | | | | | | |
| LS Mean | | | | | | | | |
| Change (vs | | | | | | | | |
| Placebo)a | | | | | | | | |

ANCOVA = analysis of covariance;
CGM = continuous glucose monitoring;
CI = confidence interval;
ITT = intent-to-treat;
LS = least squares;
Max = maximum;
Min = minimum;
SD = standard deviation
aLS Mean, its associated CI and P-value from ANCOVA with effect for treatment group and baseline value as a covariate.

TABLE 12
Change in Percentage of Time CGM Glucose Within Clinically Significant
Hypoglycemic Range (<54 mg/dL [3.0 mmol/L]) During 7-day Periods
from Baseline to End of Dosing at 100, 200, and 300 μg - ITT Population

| Percentage of time CGM Glucose in Clinically Significant Hypoglycemia | | | | | |
|---|---|---|---|---|---|
| 50 μg (Days 1 to 4)^{b} | | 100 μg (Days 5 to 11) | | 200 μg (Days 12 to 18) | |
| Placebo | MEDI0382 | Placebo | MEDI0382 | Placebo | MEDI0382 |
| N = 20 | N = 21 | N = 20 | N = 21 | N = 20 | N = 21 |

Baseline (Days −8 to −2)

| | | | | | | |
|---|---|---|---|---|---|---|
| n | 19 | 20 | 19 | 20 | 19 | 20 |
| Mean | 0.71 | 0.45 | 0.71 | 0.45 | 0.71 | 0.45 |
| (SD) | (1.42) | (0.79) | (1.42) | (0.79) | (1.42) | (0.79) |
| Median | 0.00 | 0.00 | 0.00 | 0.00 | 0.00 | 0.00 |
| (Min, Max) | (0.0, 5.1) | (0.0, 2.4) | (0.0, 5.1) | (0.0, 2.4) | (0.0, 5.1) | (0.0, 2.4) |

End of Dosing Period

| | | | | | | |
|---|---|---|---|---|---|---|
| n | 20 | 20 | 19 | 21 | 20 | 21 |
| Mean | 0.72 | 2.94 | 0.75 | 1.28 | 0.68 | 2.34 |
| (SD) | (1.38) | (4.60) | (1.84) | (1.96) | (1.20) | (3.03) |
| Median | 0.00 | 1.02 | 0.00 | 0.00 | 0.00 | 0.75 |
| (Min, Max) | (0.0, 4.3) | (0.0, 15.5) | (0.0, 7.3) | (0.0, 6.5) | (0.0, 3.9) | (0.0, 9.3) |

Change from Baseline to End of Dosing Period

| | | | | | | |
|---|---|---|---|---|---|---|
| n | 8 | 6 | 8 | 6 | 8 | 6 |
| Mean | −0.61 | 4.96 | −0.90 | 1.47 | −0.76 | −0.06 |
| (SD) | (2.30) | (5.88) | (1.35) | (2.66) | (2.51) | (2.62) |
| Median | −0.51 | 2.19 | −0.64 | 1.35 | −0.66 | −0.34 |
| (Min, Max) | (−4.0, 3.5) | (−0.3, 15.0) | (−4.0, 0.6) | (−1.5, 4.8) | (−4.0, 3.5) | (−2.4, 4.8) |
| LS Mean | −0.49 | 4.79 | −0.85 | 1.41 | −0.64 | −0.21 |
| Change^{a} | (−2.94, 1.96) | (1.96, 7.62) | (−2.07, 0.37) | (0.00, 2.81) | (−1.79, 0.51) | (−1.54, 1.12) |
| (90% CI) | | | | | | |
| Difference in | — | 5.28 | — | 2.25 | — | 0.43 |
| LS Mean | | (1.54, 9.03) | | (0.39, 4.12) | | (−1.33, 2.19) |
| Change (vs | | | | | | |
| Placebo)^{a} | | | | | | |
| (90% CI) | | | | | | |
| P-value for | — | 0.028 | — | 0.053 | — | 0.669 |
| Difference in | | | | | | |
| LS Mean | | | | | | |
| Change (vs | | | | | | |
| Placebo)^{a} | | | | | | |

| | Percentage of time CGM Glucose in Clinically Significant Hypoglycemia | | | |
|---|---|---|---|---|
| | 300 μg (Days 19 to 25) | | 300 μg (Days 26 to 32) | |
| | Placebo | MEDI0382 | Placebo | MEDI0382 |
| | N = 20 | N = 21 | N = 20 | N = 21 |
| Baseline (Days −8 to −2) | | | | |
| n | 19 | 20 | 19 | 20 |
| Mean | 0.71 | 0.45 | 0.71 | 0.45 |
| (SD) | (1.42) | (0.79) | (1.42) | (0.79) |
| Median | 0.00 | 0.00 | 0.00 | 0.00 |
| (Min, Max) | (0.0, 5.1) | (0.0, 2.4) | (0.0, 5.1) | (0.0, 2.4) |
| End of Dosing Period | | | | |
| n | 20 | 20 | 20 | 19 |
| Mean | 0.79 | 2.41 | 0.82 | 1.65 |
| (SD) | (2.00) | (4.55) | (2.20) | (2.40) |
| Median | 0.00 | 0.70 | 0.00 | 0.45 |
| (Min, Max) | (0.0, 8.2) | (0.0, 18.6) | (0.0, 8.5) | (0.0, 6.8) |
| Change from Baseline to End of Dosing Period | | | | |
| n | 8 | 6 | 8 | 5 |
| Mean | −1.10 | 3.71 | −1.46 | 0.15 |
| (SD) | (0.77) | (8.18) | (1.61) | (2.22) |
| Median | −0.66 | −0.78 | −0.66 | −0.44 |
| (Min, Max) | (−2.3, −0.4) | (−2.4, 17.5) | (−4.1 , −0.4) | (−2.4, 3.5) |
| LS Mean | −0.99 | 3.58 | −1.45 | 0.13 |
| Change^{a} | (−4.35, 2.37) | (−0.30, 7.46) | (−2.18, −0.72) | (−0.80, 1.06) |
| (90% CI) | | | | |
| Difference in | — | 4.57 | — | 1.58 |
| LS Mean | | (−0.57, 9.71) | | (0.40, 2.76) |
| Change (vs | | | | |
| Placebo)^{a} | | | | |
| (90% CI) | | | | |
| P-value for | — | 0.138 | — | 0.036 |
| Difference in | | | | |
| LS Mean | | | | |
| Change (vs | | | | |
| Placebo)^{a} | | | | |

ANCOVA = analysis of covariance;
CGM = continuous glucose monitoring;
CI = confidence interval;
ITT = intent-to-treat;
LS = least squares;
Max = maximum;
Min = minimum;
SD = standard deviation
^{a}LS Mean, its associated CI and P-value from ANCOVA with effect for treatment group and baseline value as a covariate.;
^{b}Note:
Days 1 to 4 is not 7 Days

TABLE 13
Change in Average CGM Glucose During 7-day Periods from Baseline
to End of Dosing at 100, 200, and 300 μg - ITT Population

| Glucose Over 7-Day Periods(mg/dL) | | | | |
|---|---|---|---|---|
| 100 μg (Days 5 to 11) | | 200 μg (Days 12 to 18) | | 300 μg (Days 19 to 25) |
| Placebo | MEDI0382 | Placebo | MEDI0382 | Placebo |
| N = 20 | N = 21 | N = 20 | N = 21 | N = 20 |

Baseline (Days −8 to −2)

| | | | | | |
|---|---|---|---|---|---|
| n | 18 | 19 | 18 | 19 | 18 |
| Mean | 165.004 | 165.077 | 165.004 | 165.077 | 165.004 |
| (SD) | (48.775) | (26.229) | (48.775) | (26.229) | (48.775) |
| Median | 161.605 | 165.848 | 161.605 | 165.848 | 161.605 |
| (Min, Max) | (107.24, 276.35) | (118.50, 216.45) | (107.24, 276.35) | (118.50, 216.45) | (107.24, 276.35) |

End of Dosing Period

| | | | | | |
|---|---|---|---|---|---|
| n | 18 | 19 | 20 | 21 | 17 |
| Mean | 185.010 | 127.599 | 177.927 | 131.724 | 178.425 |
| (SD) | (53.109) | (23.800) | (57.204) | (43.733) | (53.948) |
| Median | 178.200 | 122.769 | 168.599 | 121.570 | 176.971 |
| (Min, Max) | (92.40, 302.90) | (98.55, 194.98) | (102.49, 303.53) | (90.05, 279.63) | (85.80, 287.52) |

Change from Baseline to End of Dosing Period

| | | | | | |
|---|---|---|---|---|---|
| n | 16 | 17 | 18 | 19 | 15 |
| Mean | 9.612 | −35.628 | 9.692 | −30.668 | 10.064 |
| (SD) | (26.880) | (17.787) | (37.404) | (42.001) | (32.001) |
| Median | 4.073 | −31.685 | 5.049 | −32.533 | 8.331 |
| (Min, Max) | (−35.80, 58.08) | (−74.66, −8.31) | (−55.89, 90.84) | (−81.73, 98.29) | (−33.47, 73.48) |
| LS Mean | 10.38 | −36.35 | 9.68 | −30.66 | 10.22 |
| Change^{a} | (1.38, 19.38) | (−45.08, −27.62) | (−5.78, 25.14) | (−45.71, −15.61) | (−1.72, 22.15) |
| (90% CI) | | | | | |
| Difference in | — | −46.73 | — | −40.34 | — |
| LS Mean | | (−59.29, −34.16) | | (−61.91, −18.76) | |
| Change (vs | | | | | |
| Placebo)^{a} | | | | | |
| (90% CI) | | | | | |
| P-value for | — | <0.001 | — | 0.003 | — |
| Difference in | | | | | |
| LS Mean | | | | | |
| Change (vs | | | | | |
| Placebo)^{a} | | | | | |

| | Glucose Over 7-Day Periods(mg/dL) | | |
|---|---|---|---|
| | 300 μg (Days 19 to 25) | 300 μg (Days 26 to 32) | |
| | MEDI0382 | Placebo | MEDI0382 |
| | N = 21 | N = 20 | N = 21 |
| Baseline (Days −8 to −2) | | | |
| n | 19 | 18 | 19 |
| Mean | 165.077 | 165.004 | 165.077 |
| (SD) | (26.229) | (48.775) | (26.229) |
| Median | 165.848 | 161.605 | 165.848 |
| (Min, Max) | (118.50, 216.45) | (107.24, 276.35) | (118.50, 216.45) |
| End of Dosing Period | | | |
| n | 20 | 15 | 18 |
| Mean | 124.894 | 176.910 | 125.382 |
| (SD) | (24.673) | (58.571) | (29.613) |
| Median | 123.277 | 160.421 | 121.594 |
| (Min, Max) | (80.70, 207.09) | (82.36, 279.27) | (94.61, 220.54) |
| Change from Baseline to End of Dosing Period | | | |
| n | 18 | 13 | 16 |
| Mean | −38.623 | 6.564 | −41.401 |
| (SD) | (28.722) | (51.940) | (32.446) |
| Median | −35.498 | 2.924 | −51.123 |
| (Min, Max) | (−80.02, 16.60) | (−91.74, 120.58) | (−95.54, 4.09) |
| LS Mean | −38.75 | 6.52 | −41.37 |
| Change^{a} | (−49.64, −27.86) | (−12.00, 25.05) | (−58.06, −24.67) |
| (90% CI) | | | |
| Difference in | −48.97 | — | −47.89 |
| LS Mean | (−65.12, −32.81) | | (−72.83, −22.95) |
| Change (vs | | | |
| Placebo)^{a} | | | |
| (90% CI) | | | |
| P-value for | <0.001 | — | 0.003 |
| Difference in | | | |
| LS Mean | | | |
| Change (vs | | | |
| Placebo)^{a} | | | |

ANCOVA = analysis of covariance;
CGM = continuous glucose monitoring;
CI = confidence interval;
ITT = intent-to-treat;
LS = least squares;
Max = maximum;
Min = minimum;
SD = standard deviation
^{a}LS Mean, its associated CI and P-value from ANCOVA with effect for treatment group and baseline value as a covariate.

Effect of Cotadutide on Urinary Albumin Excretion and eGFR

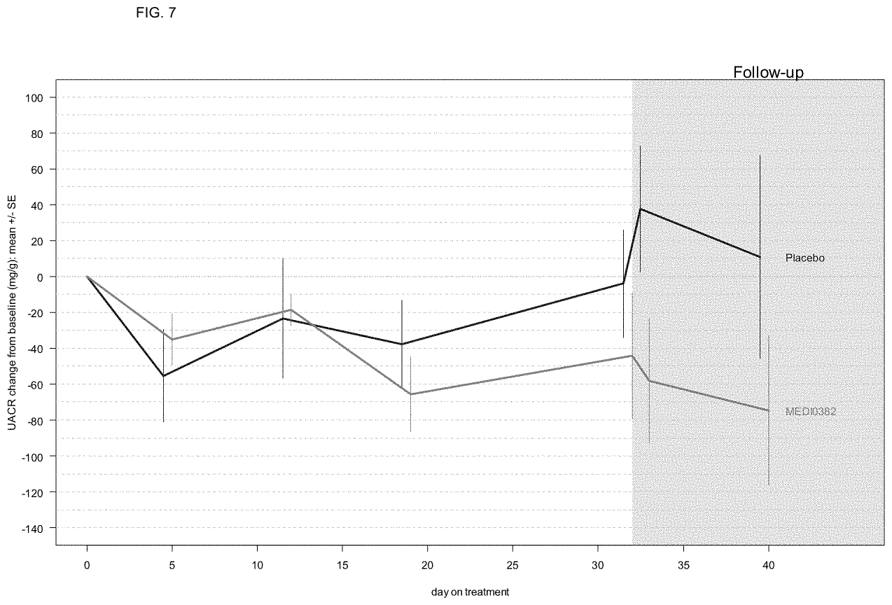

Mean urine albumin:creatinine ratio (UACR) was lower in the Cotadutide dose group compared to placebo from Day 19 until the end of dosing, and for 8 days into the follow-up period. A nonstatistically significant LS mean reduction in UACR from baseline to the end of dosing (Day 33) was observed for the Cotadutide dose group (−3.05 mg/mmol), while UACR was increased in the placebo group (by 2.02 mg/mmol) over this time period (Table 14A). The mean change of UACR from baseline in subjects with micro- or macro-albuminuria is provided in FIG. 7. The change in UACR from baseline in subjects with a mean UACR>3 mg/mmol at baseline is shown in Table 14B.

TABLE 14A
Change in Urine Albumin:Creatinine Ratio from
Baseline to End of Dosing - ITT Population

Albumin/Creatinine (mg/mmol)

| | |
|---|---|
| Placebo | MEDI0382 |
| (Total) | (Total) |
| N = 20 | N = 21 |

Baseline

| | | |
|---|---|---|
| N | 20 | 21 |
| Mean | 9.51 | 11.07 |
| (SD) | (17.49) | (23.65) |
| Median | 2.43 | 3.03 |
| (Min, Max) | (0.3, 69.5) | (0.4, 85.3) |

End of Dosing (Day 33)

| | | |
|---|---|---|
| N | 19 | 17 |
| Mean | 11.95 | 10.06 |
| (SD) | (24.17) | (22.83) |
| Median | 3.20 | 2.00 |
| (Min, Max) | (0.2, 99.3) | (0.4, 88.5) |

Change from Baseline

| | | |
|---|---|---|
| N | 19 | 17 |
| Mean | 2.02 | −3.05 |
| (SD) | (8.22) | (8.10) |
| Median | 0.00 | −0.60 |
| (Min, Max) | (−11.4, 29.8) | (−32.2, 3.2) |
| LS Mean | 2.02 | −3.05 |
| Change^{a} | (−1.20, 5.24) | (−6.46, 0.35) |
| (90% CI) | | |
| Difference | — | −5.07 |
| in LS | | (−9.77, −0.38) |
| Mean Change | | |
| (vs Placebo)^{a} | | |
| (90% CI) | | |
| P-value for | — | 0.077 |
| Difference | | |
| in LS | | |
| Mean Change | | |
| (vs Placebo)^{a} | | |

ANCOVA = analysis of covariance;
CI = confidence interval;
ITT = intent-to-treat;
LS = least squares;
Max = maximum;
Min = minimum;
SD = standard deviation
^{a}LS Mean, its associated CI and P-value from ANCOVA with effect for treatment group and value at the Baseline as a covariate.
^{b} 5/6 subjects in the Placebo Insulin Dose <20 U/day group and 6/7 subjects in the MEDI0382 Insulin Dose <20 U/day group were only on oral antidiabetic treatment.

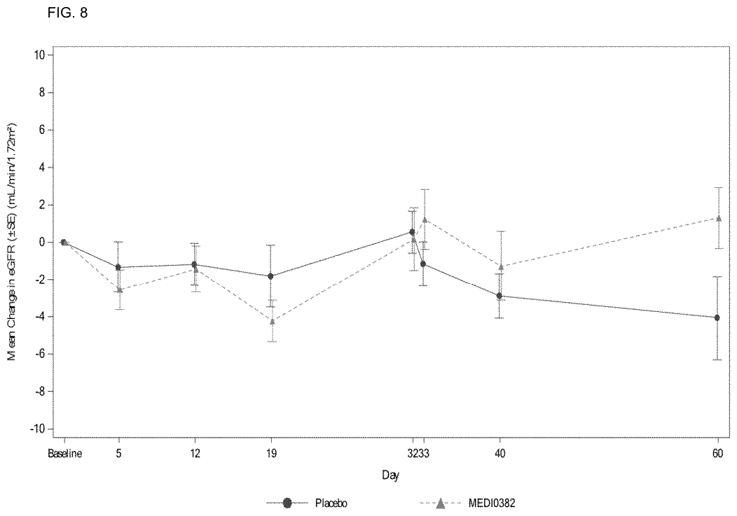

No statistically significant difference was observed in the LS mean change from baseline to the end of dosing (Day 33) between the Cotadutide group and the placebo group in terms of eGFR (FIG. 8).

TABLE 14B
Fold Change in Log-Transformed Urine Albumin:Creatinine
Ratio from Baseline to End of Dosing in Subjects with
a Mean UACR > 3 mg/mmol at Baseline-ITT Population

Albumin/Creatinine (mg/mmol)

| | |
|---|---|
| Placebo | MEDI0382 |
| (Total) | (Total) |
| N = 9 | N = 11 |

Baseline

| | | |
|---|---|---|
| N | 9 | 11 |
| Mean | 2.537 | 2.236 |
| SD | 0.946 | 1.170 |
| Median | 2.457 | 1.636 |
| (Min, Max) | (1.55, 4.24) | (1.11, 4.45) |

End of Dosing (Day 33)

| | | |
|---|---|---|
| N | 9 | 9 |
| Mean | 2.571 | 1.767 |
| SD | 1.126 | 1.508 |
| Median | 2.251 | 1.253 |
| (Min, Max) | (1.19, 4.60) | (0.00, 4.48) |
| Fold Change | | −0.70 |
| in Log- | | |
| Transformed | | |
| (vs. Placebo)^{a} | | |
| Relative Change | | 50.55% |
| from Baseline | | |
| to End of Dosing | | |
| (vs. Placebo)^{b} | | |
| Geometric Mean | 1.034 | 0.511 |
| (90% CI) | (0.674, 1.587) | (0.327, 0.800) |
| Geometric | | 0.4945 |
| Mean Ratio | | |
| (vs. Placebo) | | |
| 90% CI | | (0.277, 0.884) |
| P-value^{c} | | 0.0504 |

CI = confidence interval;
ITT = intent-to-treat;
Max = maximum;
Min = minimum;
SD = standard deviation
^{a}Mean difference of (log-transformed UACR at end of dosing − log-transformed of UACR at baseline) between MEDI0382 and placebo.
^{b}1-exponential of fold change in log-transformed (MEDI0382 vs. placebo).
^{c}P-value is from the comparison of placebo to MEDI0382 for 2-sample t-text (unequal variance with Satterthwaite approximation).

### Effect of Cotadutide on Insulin Dose Adjustment

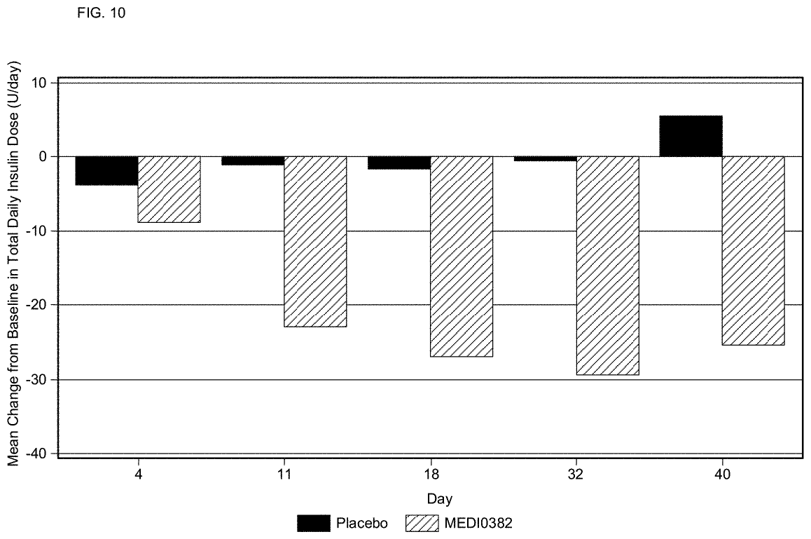

The effect of Cotadutide on insulin dose adjustment was evaluated for subjects on ≥20 U/day insulin. For all subjects on insulin, Cotadutide resulted in a greater reduction from baseline in total daily insulin dose at every study day as compared to placebo. The subset of subjects with a baseline HbA1c<8.0% showed an even greater reduction in total daily insulin dose across the dosing period compared to subjects with a baseline HbA1c ≥8.0%. The mean percent reduction in total daily insulin dose from baseline until the end of dosing was statistically significant compared to placebo, especially in the HbA1c<8.0% subgroup (Table 15). Similar results were observed in subset of subjects with a baseline HbA1c ≥8.0%, although statistical significance was not achieved. As shown in FIG. 10, the percentage reduction from baseline in the Cotadutide group at the end of treatment was −35.2% (standard deviation [SD]=44.8) compared with 0.8% (SD=21.6; p=0.012).

TABLE 15
Change in Total Daily Insulin Dose from Baseline (Day −2) to End of Dosing
(Day 32) in Subjects on Insulin Dose ≥ 20 U/day - ITT Population

| Total Daily Insulin Dose | | | | | |
|---|---|---|---|---|---|
| Placebo | MEDI0382 | Placebo | MEDI0382 | | |
| (Screening | (Screening | (Screening | (Screening | Placebo | MEDI0382 |
| HbA1c > _ 8.0%) | HbA1c > _ 8.0%) | HbA1c < 8.0%) | HbA1c < 8.0%) | (Total) | (Total) |
| N = 6 | N = 5 | N = 8 | N = 9 | N = 14 | N = 14 |

Change from Baseline to End of Dosing (U/day)

| | | | | | | |
|---|---|---|---|---|---|---|
| n | 6 | 5 | 8 | 9 | 14 | 14 |
| Mean | 0.5 | −14.0 | −1.3 | −38.0 | −0.5 | −29.4 |
| (SD) | (24.5) | (37.3) | (13.1) | (61.1) | (18.0) | (53.5) |
| Median | 6.0 | −2.0 | 0.0 | −18.0 | 0.0 | −16.0 |
| (Min, Max) | (−46, 24) | (−76, 20) | (−22, 16) | (−194, 0) | (−46, 24) | (−194, 20) |
| P-value^{a} | — | 0.481 | — | 0.113 | — | 0.073 |

Change from Baseline to End of Dosing (U/kg)

| | | | | | | |
|---|---|---|---|---|---|---|
| n | 6 | 5 | 8 | 9 | 14 | 14 |
| Mean | 0.0 | −0.2 | −0.0 | −0.4 | −0.0 | −0.3 |
| (SD) | (0.3) | (0.5) | (0.2) | (0.6) | (0.2) | (0.5) |
| Median | 0.1 | −0.0 | 0.0 | −0.2 | 0.0 | −0.2 |
| (Min, Max) | (−1, 0) | (−1, 0) | (−0, 0) | (−2, 0) | (−1, 0) | (−2, 0) |
| P-value^{a} | — | 0.444 | — | 0.108 | — | 0.065 |

Change from Baseline to End of Dosing (%)

| | | | | | | |
|---|---|---|---|---|---|---|
| n | 6 | 5 | 8 | 9 | 14 | 14 |
| Mean | 6.7 | −21.3 | −3.6 | −42.9 | 0.8 | −35.2 |
| (SD) | (25.4) | (59.9) | (18.7) | (35.7) | (21.6) | (44.8) |
| Median | 7.9 | −2.2 | 0.0 | −56.5 | 0.0 | −37.0 |
| (Min, Max) | (−35, 40) | (−100, 33) | (−34, 18) | (−100, 0) | (−35, 40) | (−100, 33) |
| P-value^{b} | — | 0.321 | — | 0.014 | — | 0.012 |

HbA1c = hemoglobin Alc;
ITT = intent-to-treat;
Max = maximum;
Min = minimum;
SD = standard deviation
^{a}P-value from 2-sample t-test (unequal variance with Satterthwaite approximation)′
^{b}P-value from 2-sample t-test (equal variance).

### Blood Pressure and Heart Rate

Based on post-hoc analysis, ABPM measurements were as follows: mean change from baseline to Day 32: −1.15 mmHg vs 2.21 mmHg (systolic, Cotadutide vs Placebo) and 2.54 mmHg vs 1.84 mmHg (diastolic, Cotadutide vs Placebo). Increases in supine blood pressure from baseline were also observed after 32 days of treatment: 1.44 mmHg vs 8.69 mmHg (systolic, Cotadutide vs placebo) and 0.57 mmHg vs 3.40 mmHg (diastolic, Cotadutide vs placebo); however, these changes were also not clinically significant and were numerically greater in the placebo group.

Based on office-based measurement, increases in postural blood pressure difference were observed after 32 days of treatment: 8.9 mmHg vs 0 1 mmHg (systolic, Cotadutide vs placebo) and 0.8 mmHg vs 1 8 mmHg (diastolic, Cotadutide vs placebo); however, these changes were not clinically significant.

Based on in-clinic assessments and ABPM, an increase in heart rate was observed in the Cotadutide dose group starting on Day 5. By Day 32, the LS mean change from baseline in pulse rate (office-based measure) was an increase of 14.13 bpm for subjects in the Cotadutide dose group and 3.14 bpm for subjects in the placebo dose group (p <0.001). Post-hoc analysis (performed following removal of inadequate quality reports) of 24-hour pulse rate data recorded by ABPM showed a mean increase in heart rate from baseline to Day 32 by 11.85 bpm for subjects in the Cotadutide dose group compared to −0.92 bpm for subjects in the placebo dose group. Mean heart rate changes from baseline to Day 32 were numerically greater while asleep (13.06 bpm vs −2.28 bpm) compared to awake (10.78 bpm vs −0.80 bpm) for subjects on Cotadutide vs placebo, respectively. By the end of the study (Day 60), no meaningful differences in heart rate from baseline were observed.

### Effect of Cotadutide on Fluid Status

There were no clinically meaningful differences between Cotadutide and placebo in terms of changes in total body water volume, intracellular fluid volume, or extracellular fluid volume from baseline (Day 1) to the end of dosing using bioimpedance spectroscopy.

### Pharmacokinetic Parameters

The endpoints of maximum observed concentration (Cₘₐₓ), tₘₐₓ, and AUC₀₋₂₄ on Day 32 following Cotadutide doses of 300 μg were assessed; trough plasma concentration (C_{trough}) at other dose levels were evaluated on Days 5, 12, 19, 32, and 33 to check for exposure during the study.

Pharmacokinetic characterization of Cotadutide at 300 μg was obtained using full PK profiles collected during the last day of dosing at 300 μg (Day 32). Additional C_{trough} concentrations were collected through the study duration to check exposure.

Overall, PK data collected in this study suggest that repeat daily SC administration of Cotadutide doses ranging from 50 to 300 μg show linear PK for C_{trough}, with mean Cₘₐₓ at 300 μg of 16.93 ng/mL and median tₘₐₓ of 5.6 hours, in agreement with data from subjects without renal impairment.

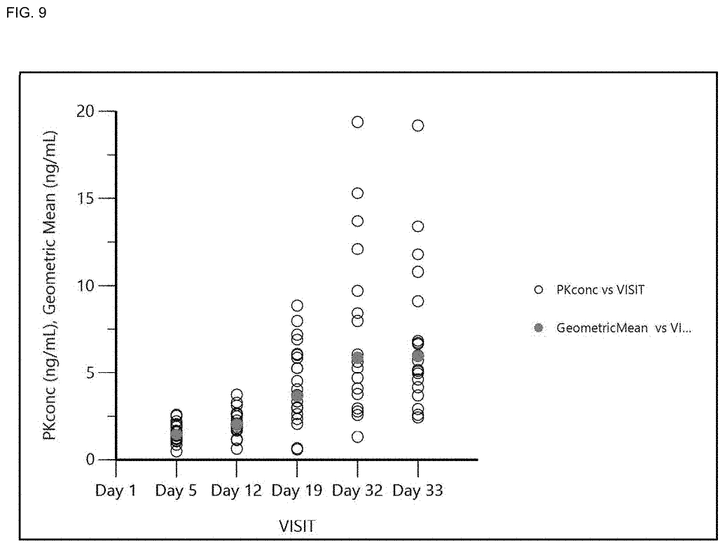

A summary of the main PK parameters is presented in Table 16, and C_{trough} exposure is presented in FIG. 9.

TABLE 16
Summary of PK Parameters for MEDI0382
on Day 32 After Subcutaneous Dose at
300 μg (Geometric Mean [Range])

| | Dose | Day of | Cₘₐₓ | tₘₐₓ | AUC_{τ} |
|---|---|---|---|---|---|
| Cohort | (μg) | Dose | (ng/mL) | (hr)^{a} | (ng · hr/mL) |
| 1 | 300 | 32 | 16.93 | 5.6 | 285.93 |
| (N = 18) | | | [5.17-35.4] | [4-24] | [124.08-669.17] ^{b} |

AUC_{τ} = area under the concentration-time curve during the dosing interval;
Cₘₐₓ = maximum observed concentration;
N = number of values included in the statistical analysis;
tₘₐₓ = time to Cₘₐₓ
^{a}Median and range
^{b} N = 16

### Immunogenicity

Development of ADA and titres (in any subjects testing positive) were assessed (As-treated population). No subjects tested positive for ADA at baseline. A total of 2 subjects (4.9%) were ADA positive post-baseline (both subjects [9.5%] in the Cotadutide dose group). No subjects had a treatment-boosted ADA, defined as baseline ADA titer that was boosted to a 4-fold or higher level during drug administration. 1 subject was ADA positive at Day 12 (titer ≤5) but negative at subsequent visits. One subject was ADA positive at Day 32 (titer=2560) and remained positive at the Day 60 follow-up visit (titer=160); at 6 months after their last visit; the antibody titer was 10, and therefore, no additional follow-up was deemed necessary for this subject.

There were no adverse events (AEs) recorded in association with positive ADAs.

### Safety/Adverse Events

There was one death in this study due to diabetic ketoacidosis and cholecystitis that was deemed related to Cotadutide; however, no clear indicators of relationship to Cotadutide were identified.

Serious adverse events (SAEs) were balanced between the placebo and Cotadutide groups.

Three subjects reported a treatment-emergent adverse events (TEAE) that led to withdrawal from Cotadutide/placebo; two subjects were assigned to the Cotadutide group and one was assigned to the placebo group. The incidence of overall TEAEs and Cotadutide/product-related TEAEs was higher in the Cotadutide dose group compared with the placebo dose group and were largely driven by gastrointestinal side-effects. Overall, the most frequently reported TEAEs at the preferred term (PT) level for subjects in the Cotadutide dose group were: nausea, vomiting, diarrhea, and dyspepsia. The majority of nausea and vomiting events were mild (Grade 1) in severity; one subject experienced severe (Grade 3) nausea and vomiting. All events were non-serious, transient in nature, did not result in study discontinuation, and had resolved by the end of the study.

There were no severe events of hypoglycemia requiring third party assistance or hospitalization.

There was one adverse event (AE) of glomerular filtration rate decreased which led to Cotadutide/placebo discontinuation. This AE arose in conjunction with nausea and vomiting in a subject who was taking three different diuretics.

Subjects on Cotadutide showed an increase in pulse rate while on treatment (the magnitude of which was greater while asleep compared to awake) and no clinically or statistically significant changes in blood pressure.

### CONCLUSIONS

A statistically significant reduction from baseline to end of treatment in percentage plasma glucose AUC_{0-4 h} during MMTT was observed for the Cotadutide dose group, titrated up to a dose level of 300 μg, compared with the placebo dose group.

Glycemic control was achieved in subjects on Cotadutide during the treatment period, as evidenced by significant reductions in HbA1c and increased time within a target glucose range 70 mg/dL to 180 mg/dL on CGM as compared to placebo. Numerical reductions were also observed in fasting glucose.

A statistically significant LS mean reduction from Day 1 to Day 33 in absolute body weight was observed in the Cotadutide dose group compared with the placebo dose group.

A total of 2 subjects in the Cotadutide dose group (9.5%) developed treatment-emergent ADA over the course of the study; one subject remained positive at the end of study and at 6 months after their last visit; the antibody titer at 6 months was 10, and therefore, no additional follow-up was deemed necessary for this subject.

Subjects on Cotadutide spent a significantly greater percentage of time within the target glucose range of 70-180 mg/dL, less percentage of time above 180 mg/dL, and had significantly reduced average glucose levels in comparison to placebo at all dose levels. Numerical reductions in coefficient of variation, reflective of improved glycemic variability, were also observed.

A small imbalance was observed for percentage time spent in hypoglycemia for the Cotadutide group vs placebo. As dosing progressed and insulin dose adjustments were made, the % of time spent in hypoglycemic range decreased and was not clinically or statistically significant from placebo at the end of dosing.

Subjects on Cotadutide had a numerical reduction in urine albumin:creatinine ratio (UACR) compared to placebo over the course of the dosing period.

No significant effects of Cotadutide on eGFR or body water volume were found.

Significant and sizeable reductions in insulin dose requirement were observed in the Cotadutide group during the study. Subjects with a baseline HBA1c <8.0% experienced greater reductions in insulin.

Repeat daily SC administration of Cotadutide doses ranging from 50 to 300 μg showed linear PK for C_{trough}, and daily exposure at 300 μg comparable to that observed in subjects without renal impairment.

### Example 3: Clinical Evaluation of Cotadutide in Patients with T2DM and Renal Impairment (Phase 2b)

A Phase 2b randomized, placebo-controlled, double-bind study is performed to further demonstrate the efficacy and safety of Cotadutide in subjects with Type 2 Diabetes Mellitus (T2DM) and Diabetic Kidney Disease (DKD).

### (A) Subjects

The study enrolls subjects with T2DM and Diabetic Kidney Disease (DKD) (eGFR ≥20 and <90 mL/min/1.73 m² and micro- or macroalbuminuria). Approximately 593 participants are screened/enrolled to achieve 237 randomly assigned to study intervention and 192 participants who complete study treatments.

The subjects are screened for the following inclusion and exclusion criteria.

Inclusion Criteria:
Male and female subjects, age ≥18 and ≤79 years at the time of signing the informed consent;
eGFR ≥20 to ≤90 mL/min/1.73 m² determined at the screening visit or a documented occurrence at least 3 months prior to randomization. eGFR is determined using the chronic kidney disease epidemiology collaboration equation (CKD-EPI). Rescreening on eGFR may be repeated twice;
Receiving background standard of care treatment for renal disease and/or T2DM and being treated according to locally recognised guidelines, as appropriate;
Receiving optimized and stable treatment with an angiotensin-converting-enzyme (ACE) inhibitor or an angiotensin II receptor antagonist for ≥3 months at screening at the maximum tolerated dose (MTD) unless contraindicated, not tolerated, or not practically available or suitable. Participants who cannot tolerate an ACE inhibitor or an angiotensin II receptor antagonist may still be eligible to enter the study.
Micro- or macroalbuminuria as defined by UACR >50 mg/g or 5.7 mg/mmol. Rescreening on UACR may be repeated twice;
Diagnosed with T2DM with glucose control managed with any insulin and/or any oral therapy combination including metformin, SGLT2 inhibitor, thiazolidinedione, or acarbose where no major dose changes (e.g., >50%) have occurred within the 4 weeks prior to the start of the run-in period. Participants taking sulfonylureas or glitinides can be randomised following a 4-week washout period of the sulfonylurea/glitinide;
At least 40% of participants should be on an SGLT2 inhibitor that was initiated at least 4 weeks prior to screening;
HbA1c range of 6.5% to 12.5% (inclusive) at screening;
Body mass index (BMI) >25 kg/m² at screening or >23 kg/m² for participants enrolled in Japan

Exclusion Criteria:
History or presence of significant medical or psychological conditions, including significant abnormalities in laboratory parameters or vital signs including ECG, which would compromise the participant's safety or successful participation in the study;
Receiving renal replacement therapy or expected to require it within 6 months of being randomized;
Renal transplant or on the waiting list for renal transplantation;
History of substance dependence, alcohol abuse, or excessive alcohol intake;
Received a GLP-1 analogue-containing preparation within the last 30 days or 5 half-lives of the drug (if known; whichever is longer) at the time of Visit 2
Received any of the following medications within the specified time frame prior to the start of the study (Visit 2): (a) Aspirin (acetylsalicylic acid) at a dose greater than 150 mg once daily and within the last 3 days prior to the start of the run-in period (Visit 2), (b) Paracetamol (acetaminophen) or paracetamol-containing preparations at a total daily dose of greater than 3000 mg and within the last 3 days prior to the start of the run-in period (Visit 2), or (c) Ascorbic acid (vitamin C) supplements at a total daily dose of greater than 1000 mg and within the last 3 days prior to the start of the run-in period (Visit 2);
Symptoms of acutely decompensated blood glucose control (e.g., thirst, polyuria, weight loss) or recent episodes of severe hypoglycemia;
T1DM, history of diabetic ketoacidosis, or clinical suspicion of type 1 diabetes mellitus (T1DM) (eg, undetectable levels of C peptide and positive tests for antibodies indicative of T1DM);
Participants with recent acute or subacute renal function deterioration (e.g., participants with large fluctuations of creatinine values documented within the 3 months prior to screening;
Significant inflammatory bowel disease, gastroparesis, or other severe disease or surgery affecting the upper GI tract (including weight-reducing surgery and procedures) which may affect gastric emptying or could affect the interpretation of safety and tolerability data;
History of acute or chronic pancreatitis;
Significant hepatic disease (except for non-alcoholic steatohepatitis or nonalcoholic fatty liver disease without portal hypertension or cirrhosis) and/or subjects with any of the following results:
   Aspartate transaminase (AST) ≥3×upper limit of normal (ULN);
   Alanine transaminase (ALT) ≥3×ULN; or
   Total bilirubin ≥2×ULN
Poorly controlled hypertension defined as (a) Systolic BP >180 mm Hg (b) Diastolic BP ≥100 mm Hg after 10 minutes of seated rest and confirmed by repeated measurement at screening. Subjects who fail BP screening criteria can be considered for 24-hour ambulatory blood pressure monitoring (ABPM). Subjects who maintained a mean 24-hour systolic BP≤180 or diastolic BP<100 mm Hg with a preserved nocturnal dip of >15% are considered eligible;
Unstable angina pectoris, myocardial infarction, transient ischemic attack or stroke within 3 months prior to screening, or participants who have undergone percutaneous coronary intervention or a coronary artery bypass graft within the past 6 months or who are due to undergo these procedures at the time of screening
Decompensated heart failure or hospitalisation for heart failure within 3 months prior to screening or symptoms consistent with New York Heart Association heart failure Class III or IV;
Basal calcitonin level >50 ng/L at screening or history/family history of medullary thyroid carcinoma or multiple endocrine neoplasia;
History of neoplastic disease within 5 years prior to screening, except for adequately treated basal cell skin cancer, squamous cell skin cancer, or in situ cervical cancer.

The 237 participates are randomized into 2 cohorts: 225 participants in Cohort 1 and 12 participants in Cohort 2. (Cohorts 1 and 2 are described below in the “Study Design.”)

### (B) Study Design

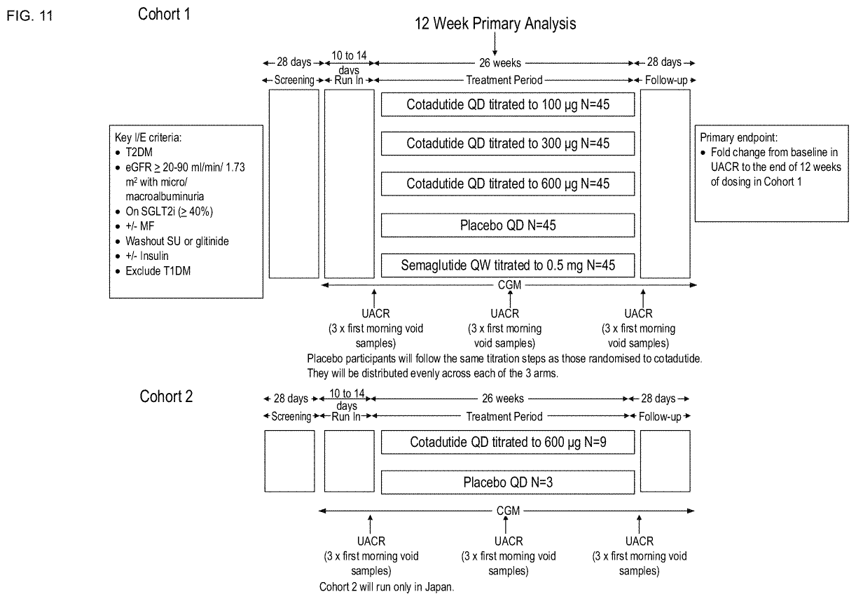

A flow diagram of the proposed study is provided in FIG. 11. Cotadutide is titrated from 50 μg up to 600 μg (50, 100, 300, or 600 μg) and administered once daily by subcutaneous (SC) injection over 26 weeks. The open-label active comparator, semaglutide, is administered SC from 0.25 to 0.5 mg once weekly over 26 weeks. Cotadutide, placebo, and semaglutide are administered using an injection pen device. The study is conducted in two cohorts.

Cohort 1 randomizes approximately 225 participants at multiple sites in approximately 3 countries. Participants are randomised in a 1:1:1:1:1 ratio to 1 of 3 cotadutide arms (100, 300, or 600 μg, following different periods of titration), placebo, or an open-label semaglutide arm (0.5 mg). Each cotadutide arm is placebo-matched with respect to titration schedule and dose levels. Both cotadutide and placebo arms are double-blinded, and both are administered subcutaneously (SC) once daily for a total of 26 weeks. For participants assigned to cotadutide or placebo, doses commence at 50 μg and are uptitrated every two weeks to a final dose of either 100, 300, or 600 μg. Semaglutide is administered SC once weekly for a total of 26 weeks. Japanese participants in Cohort 1 will not be randomised to the 600 μg cotadutide arm. For Cohort 1, study treatments are titrated in discrete steps as shown in Table 17A or 17B.

TABLE 17A
Titration Regimen for Cohort 1 (Option 1)

| | Treatment Arm (N) | | | | |
|---|---|---|---|---|---|
| | Weeks - Dose | Weeks - Dose | Weeks - Dose | Weeks - Dose | Weeks - Dose |
| Cotadutide | 1 and 2 - 50 μg | 3 to 26 - 100 μg | | | |
| 100 μg (45) | | | | | |
| Cotadutide | 1 and 2 - 50 μg | 3 and 4 - 100 μg | 5 and 6 - 200 μg | 7 to 26 - 300 μg | |
| 300 μg (45) | | | | | |
| Cotadutide | 1 and 2 - 50 μg | 3 and 4 - 100 μg | 5 and 6 - 200 μg | 6 and 8 - 400 μg | 9 to 26 - 600 μg |
| 600 μg (45)^{a} | | | | | |

| Placebo (45) | 26 - placebo ^{b} | | |
|---|---|---|---|
| Semaglutide | 1 to 4 - 0.25 mg | 5 to 8 - 0.5 mg | 9 to 26 - 0.5 mg |
| 0.5 mg (45) | | | |

N = number of participants
^{a}Japanese participants randomized in Cohort 1 at sites in Japan will not be randomized to the 600 μg cotadutide arm.
^{b} Participants randomized to placebo will follow 1 of the 3 titration regimens matched to the cotadutide treatment arms and are distributed evenly across the placebo arms.

TABLE 17B
Titration Regimen for Cohort 1 (Option 2)

| | Treatment Arm (N) | | | | |
|---|---|---|---|---|---|
| | Weeks - Dose | Weeks - Dose | Weeks - Dose | Weeks - Dose | Weeks - Dose |
| Cotadutide | 1 and 2 - 50 μg | 3 to 26 - 100 μg | | | |
| 100 μg (45) | | | | | |
| Cotadutide | 1 and 2 - 50 μg | 3 through 6 - 100 μg | 7 and 8 - 200 μg | 9 to 26 - 300 μg | |
| 300 μg (45) | | | | | |
| Cotadutide | 1 and 2 - 50 μg | 3 through 6 - 100 μg | 7 and 8 - 200 μg | 9 and 10 - 400 μg | 11 to 26 - 600 μg |
| 600 μg (45)^{a} | | | | | |

| Placebo (45)^{a}, ^{b} | 26 - titration regimen matched to Cotadutide | | |
|---|---|---|---|
| Semaglutide | 1 to 4 - 0.25 mg | 5 to 8 - 0.5 mg | 9 to 26 - 0.5 mg |
| 0.5 mg (45)^{a} | | | |

N = number of participants
^{a}Japanese participants randomized in Cohort 1 at sites in Japan are not be randomized to the 600 μg cotadutide arm.
^{b} Participants randomized to placebo follow 1 of the 3 titration regimens matched to the cotadutide treatment arms and are distributed evenly across the placebo arms.

Cohort 2 randomizes approximately 12 Japanese participants at multiple sites in Japan. The Japanese participants are randomised in a 3:1 ratio to either cotadutide (up-titrated from 50 μg to 600 μg) or placebo for a total of 26 weeks following the titration. Titration steps in the placebo group will mimic those followed by participants randomized to cotadutide 600 μg. Both cotadutide and placebo arms are double-blinded. Cohort 2 follows the titration schedule shown in Table 18A or 18B.

TABLE 18A
Titration Regimen for Cohort 2 (Option 1)

| | Treatment Arm (N) | | | | |
|---|---|---|---|---|---|
| | Week - Dose | Week - Dose | Week - Dose | Week - Dose | Week - Dose |
| Cotadutide | 1 to 2 - 50 μg | 3 to 4 - 100 μg | 5 to 6 - 200 μg | 6 to 8 - 400 μg | 9 to 26 - 600 μg |
| 600 μg (12) | | | | | |

| | |
|---|---|
| Placebo (3) | 26 - placebo |

N = number of participants

TABLE 18B
Titration Regimen for Cohort 2 (Option 2)

| | Treatment Arm (N) | | | | |
|---|---|---|---|---|---|
| | Week - Dose | Week - Dose | Week - Dose | Week - Dose | Week - Dose |
| Cotadutide | 1 to 2 - 50 μg | 3 through 6 - 100 μg | 7 and 8 - 200 μg | 9 and 10 - 400 μg | 11 to 26 - 600 μg |
| 600 μg (12) | | | | | |

| | |
|---|---|
| Placebo (3) | 26 - placebo |

N = number of participants

The randomization is stratified according to whether a participant is on an SGLT2 inhibitor therapy at screening or not.

Both Cohorts 1 and 2 have a 14-day run-in period of diet and exercise and continuous glucose monitoring (CGM) followed by a 26-week treatment period and 28-day follow-up period.

Once daily dosing with cotadutide or placebo begins on Day 1 at 50 μg, and participants follow the titration regimen to which they have been randomized Participants randomized to the semaglutide arm receive once weekly doses also beginning on Day 1 at a starting dose of 0.25 mg once weekly and following the titration regimen detailed in the label, reaching 0.5 mg once weekly after 8 weeks.

In the event a participant randomized to a blinded arm in either cohort experiences a significant vomiting event, the dose level can be reduced to the previous titration step for 7 days before resuming the up-titration regimen. This adjustment can occur up to a maximum of two times.

UACR is measured following three first morning void collections at home prior to the clinic visit during the run in and on Days 85 and 182. All other UACR calculations are determined from single urine samples taken in the clinic.

Urine and blood samples to measure UACR, HbA1c, and fasting glucose are taken throughout the study. Assessments via continuous glucose monitoring and UACR or creatinine monitoring are also performed. Weight measurements and ECG are also performed. Plasma samples are collected for measurement of cotadutide concentrations.

Insulin dose reductions are considered for any participant at risk of hypoglycaemia. A 30% reduction in insulin dose is made from Day −1 for participants in the cotadutide or placebo arm taking insulin who has a screening HbA1c of <8.0% and eGFR of <50 mL/min/1.73 m²; this reduced dose is continued for the remainder of the study or until insulin dose titration is necessary. A 20% reduction in insulin dose is made for participants in the cotadutide or placebo arm with screening HbA1c ≥8 and eGFR ≥50 mL/min/1.73 m².

### (C) Efficacy Assessments

The percent change in UACR from baseline to the end of 12 weeks of treatment is analyzed using an analysis of covariance (ANCOVA) model with a two-sided significance level of 0.05 in order to demonstrate that Cotadutide decreases UACR in participants with diabetic kidney disease and T2DM. The change is also measured at the end of 14 weeks. The model includes fixed effect of treatment and the baseline value as well as the stratification factor (whether a participant is on an SGLT2 inhibitor therapy at screening or not) as covariates. A similar analysis is used to demonstrate that Cotadutide decreases UACR in participants with diabetic kidney disease and T2DM after 26 weeks of treatment.

A comparison of HbA1c levels from baseline to the end of 12 weeks and 26 weeks of treatment in participants receiving Cotadutide vs placebo is performed to demonstrate that Cotadutide results in decreased HbA1c in participants with diabetic kidney disease and T2DM. The change is also measured at the end of 14 weeks.

A comparison of fasting glucose levels from baseline to the end of 12 weeks and 26 weeks of treatment in participants receiving Cotadutide vs placebo is performed to demonstrate that Cotadutide results in decreased fasting glucose levels in participants with diabetic kidney disease and T2DM. The change is also measured at the end of 14 weeks.

A comparison of eGFR (calculated using creatinine and cystatin C in the CKD-epidemiology collaboration (CKD-EPI) equation (percentage and absolute change in eGFR and change in total eGFR slope) from baseline to the end of the 26 weeks of treatment in participants receiving Cotadutide vs placebo is performed to demonstrate that Cotadutide does not adversely affect eGFR and can reduce decline in eGFR.

A comparison of the weight loss from baseline to the end of 26 weeks of treatment in participants receiving Cotadutide vs placebo is performed to demonstrate that Cotadutide results in weight loss in participants with diabetic kidney disease and T2DM.

### (D) Results

Clinically meaningful and statistically significantly results were observed in an evaluation of 41 patients with T2DM (HbA1c: ≥6.5-≤10.5%) and chronic kidney disease (CKD) stage G3 (estimated glomerular filtration rate [eGFR]: ≥30-<60 mL/min/1.73 m2), on insulin and/or oral therapy, with a BMI of 25-45 kg/m². Over 32 days, 21 patients received once-daily subcutaneous cotadutide (n=21) titrated up to 300 μg, and 20 patients received placebo (PBO). In these patients, Cotadutide significantly reduced MMTT glucose AUC vs. baseline (−26.7%, 90% CI: −34.6 to −18.8) and vs PBO (3.7%, 90% CI: −3.8 to 11.2; P<0.001), with a 35.2% reduction in insulin dose (P=0.012). Cotadutide significantly reduced body weight (BW) (−3.7%) and HbA1c (−0.7%; both P<0.001). After 32 days of cotadutide treatment, no significant changes were observed in eGFR or blood pressure. C-peptide levels in the cotadutide group increased significantly vs PBO (LS mean change: 0.88 μg/L, 90% CI: 0.57 to 1.19, P<0.001). In patients with baseline micro- or macroalbuminuria (n=18), UACR was reduced by 50.6% vs PBO (P=0.0504). Serious adverse events (AEs) were balanced between treatment arms; treatment-related AEs were more frequent with cotadutide (71%) vs PBO (35%). The most common AEs were nausea (cotadutide, 33%; PBO, 20%) and vomiting (cotadutide, 24%; PBO, 5%). Pulse rate was significantly increased (11 beats per minute; P<0.001) by day 32. Thus, in patients with T2DM and chronic kidney disease, cotadutide improved overall glycemic control and glucose responses to an MMTT with acceptable tolerability. Improvements in albuminuria indicate that cotadutide can slow long-term progression of CKD.

The disclosure is not to be limited in scope by the specific embodiments described which are intended as single illustrations of individual aspects of the disclosure, and any compositions or methods which are functionally equivalent are within the scope of this disclosure. Indeed, various modifications of the disclosure in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference.

1. A method of treating chronic kidney disease (CKD) in a human patient, the method comprising administering to the patient a sufficient amount of Cotadutide (SEQ ID NO:4) to treat the CKD.

2. A method of decreasing urine albumin:creatinine ratio (UACR) in a human patient with CKD, the method comprising administering to the patient a sufficient amount of Cotadutide (SEQ ID NO:4) to decrease UACR.

3. A method of reducing body weight in a human patient with CKD, the method comprising administering to the patient a sufficient amount of Cotadutide (SEQ ID NO:4) to reduce body weight.

4. A method of improving glycemic control in a human patient with CKD, the method comprising administering to the patient a sufficient amount of Cotadutide (SEQ ID NO:4) to improve glycemic control.

5. The method of claim 1, wherein the cotadutide is administered at an initial dose of at least 20 μg daily, optionally a dose of about 50 μg daily, and then administered at a second higher dose.

6. The method of claim 5, wherein the cotadutide is administered at a third dose after the administration of the second dose, wherein the third dose is higher than the second dose, optionally wherein the third dose does not exceed 600 μg daily or wherein the third dose does not exceed 300 μg daily.

7. The method of claim 5, wherein the cotadutide is administered at a third dose after the administration of the second dose, optionally a fourth dose after the administration of the third dose, and optionally a fifth dose after the fourth dose, wherein the third dose is higher than the second dose, the fourth dose, when present, is higher than the third dose, the fifth dose, when present, is higher than the fifth dose, and the sixth dose, when present, is higher than the fourth dose.

8. The method of claim 5, wherein the initial dose is administered daily for about 4 days to about 14 days.

9. The method of claim 5, wherein the dose of cotadutide does not exceed 600 μg daily or does not exceed 300 μg daily.

10. The method of claim 5, wherein the cotadutide is administered at an initial dose of 50 μg daily for 14 days and then at a second dose of 100 μg daily.

11. The method of claim 10, wherein the cotadutide is administered at the second dose of 100 μg daily for 14 days or 28 days and then at a third dose of 200 μg daily.

12. The method of claim 11, wherein the cotadutide is administered at the third dose of 200 μg daily for 14 days and then at a fourth dose of 400 μg daily.

13. The method of claim 12, wherein the cotadutide is administered at the fourth dose of 400 μg daily for 14 days and then at a fifth dose of 600 μg daily.

14. The method of claim 5, wherein the cotadutide is administered at an initial dose of 50 μg daily for 4 days and then at a second dose of 100 μg daily.

15. The method of claim 14, wherein the cotadutide is administered at the second dose of 100 μg daily for 7 days and then at a third dose of 200 μg daily.

16. The method of claim 15, wherein the cotadutide is administered at the third dose of 200 μg daily and then at a fourth dose of 300 μg daily.

17. The method of claim 5, wherein the cotadutide is administered at an initial dose of 100 μg daily for 7 days, at second dose of 200 μg daily for the next 7 days, and subsequently at a dose of 300 μg daily.

18. The method of claim 5, wherein the cotadutide is administered by injection, optionally wherein the administration is subcutaneous.

19. The method of claim 1, wherein the administration reduces the mixed-meal tolerance test (MMTT) plasma glucose area under the curve (AUC)_{0-4 hours} in the patient, optionally wherein the administration reduces the MMTT plasma glucose AUC by at least 15%, by at least 20%, or by at least 25%.

20. The method of claim 1, wherein the administration reduces Hemoglobin A1c (HbA1c) in the patient.

21. The method of claim 1, wherein the administration reduces fasting plasma glucose (FPG) in the patient.

22. The method of claim 1, wherein the administration reduces continuous glucose monitoring (CGM) glucose AUC₀₋₂₄ in the patient.

23. The method of claim 1, wherein the administration reduces hyperglycemic glucose levels in the patient.

24. The method of claim 1, wherein the patient was using insulin prior to the administration of the Cotadutide and the administration of the Cotadutide reduces insulin use by the patient.

25. The method of claim 1, wherein the reduction occurs with 3 weeks, within 12 weeks, within 14 weeks, or within 26 weeks from the initial administration of the Cotadutide.

26. The method of claim 1, wherein the administration increases the amount of time the patient has euglycemic glucose levels, optionally wherein the amount of time is measured over a 7-day period.

27. The method of claim 1, wherein the administration improves insulin resistance in the patient, optionally wherein the insulin resistance is measured using the Homeostatic Model Assessment of Insulin Resistance (HOMA-IR) and/or the MATSUDA index.

28. The method of claim 1, wherein the administration improves beta cell function in the patient.

29. The method of claim 1, wherein the administration treats CKD in the patient.

30. The method of claim 1, wherein the administration decreases the urine albumin:creatinine ratio (UACR) of the patient, optionally wherein the administration decreases the UACR by at least 40%, by at least 45%, or by at least 50%.

31. The method of claim 1, wherein the administration reduces body weight of the patient.

32. The method of claim 31, wherein the administration reduces the body weight of the patient by at least 3%, by at least 5%, or by at least 10%.

33. The method of claim 1, wherein the administration improves glycemic control in the patient.

34. The method of claim 1, wherein the administration is for at least two weeks, for at least 12 weeks, for at least 14 weeks, or for at least 26 weeks.

35. The method of claim 1, wherein the administration is an adjunct to diet and exercise.

36. The method of claim 1, wherein the patient has an estimated glomerular filtration rate (eGFR) of <90 mL/min/1.73 m² prior to the administration.

37. The method of claim 36, wherein the patient has an eGFR <60 mL/min/1.73 m² prior to the administration.

38. The method of claim 37, wherein the patient has an eGFR ≥20 mL/min/m² prior to the administration.

39. The method of claim 38, wherein the patient has an eGFR ≥30 mL/min/m² prior to the administration.

40. The method of claim 1, wherein the patient has micro- or macro-albuminuria.

41. The method of claim 1, wherein the patient has an HBA1c<8.0% prior to the administration

42. The method of claim 1, wherein the patient has a body mass index (BMI) of ≥23 kg/m² or ≥25 kg/m² prior to the administration.

43. The method of claim 1, wherein the patient has a BMI ≤40 kg/m² prior to the administration.

44. The method of claim 1, wherein the patient has UACR>3 mg/mmol prior to the administration.

45. The method of claim 1, wherein the human patient with CKD has diabetes.

46. The method of claim 45, wherein the diabetes is type 2 diabetes.

47. The method of claim 1, wherein the human patient with CKD does not have diabetes.

48-92. (canceled)
